(19) United States
(12) Patent Application Publication
(10) Pub. No.: US 2003/0236458 A1
(43) Pub. Date: Dec. 25, 2003

(54) Spectroscopic systems and methods for detecting tissue properties

(75) Inventor: Daryl W. Hochman, Bahama, NC (US)

Correspondence Address: SPECKMAN LAW GROUP, 1501 WESTERN AVE, SUITE 100, SEATTLE, WA, 98101, US

(73) Assignee: BIOPHYSICA LLC, Seattle, WA

(21) Appl. No.: 10/374,157

(22) Filed: Feb. 24, 2003

Related U.S. Application Data
(63) Continuation-in-part of application No. 09/366,501, filed on Aug. 3, 1999, now abandoned.
(63) Continuation-in-part of application No. 09/368,087, filed on Aug. 3, 1999, now abandoned.
(63) Continuation-in-part of application No. 09/368,257, filed on Aug. 3, 1999, now abandoned.

(51) Int. Cl. A61B 6/00

(52) U.S. Cl. 600/431

(57) ABSTRACT

Methods for optically detecting physiological properties in an area of interest by detecting changes in the intrinsic or extrinsic optical properties of tissue in the area of interest are disclosed. The present invention optically detects blood flow changes, blood characteristics and blood vessel abnormalities, as well as determining the presence and location of abnormal or pathological tissue for identifying and mapping the margins of abnormal tissue, such as tumor tissue during surgical or diagnostic procedures, and for grading and characterizing tumor tissue. The present invention also provides systems and methods for distinguishing neuronal tissue from surrounding tissue, for distinguishing functional neuronal tissue from dysfunctional tissue, and for imaging functional neuronal areas in the cortex. Methods and systems of the present invention may be implemented using a contrast enhancing agent or by stimulation of activity.

### CROSS REFERENCE TO RELATED APPLICATION

This patent application is a continuation-in-part of patent application Ser. No. 09/366,501 filed on Aug. 3, 1999; a continuation-in-part of patent application Ser. No. 09/368,087 filed on Aug. 3, 1999; and a continuation-in-part of patent application Ser. No. 09/368,257 filed on Aug. 3, 1999, all of which are incorporated herein by reference in their entireties.

### TECHNICAL FIELD OF THE INVENTION

Methods and systems of the present invention relate to detection and monitoring of blood characteristics, blood flow, blood accumulation and blood vessels using optical, spectroscopic detection techniques. The methods and systems have applications for diagnosing various conditions and disease states characterized by abnormal blood characteristics, blood flow, blood accumulation and blood vessels, and for monitoring such conditions.

The present invention also relates to methods and systems for detecting abnormalities, such as cancer and pathological conditions, in cells and tissues using optical, spectroscopic techniques. More specifically, the methods and apparatus of the present invention relate to the use of contrast enhancing agents in connection with optical spectroscopic techniques to distinguish abnormal or pathological tissue, such as cancerous tissue, from normal tissue and to grade and characterize cancerous tissue.

The present invention further relates to methods and systems for detecting cellular and tissue properties in the central and peripheral nervous systems using optical, spectroscopic detection techniques. The methods and systems of the present invention relate, more specifically, to optical detection and mapping of functional neuronal activity, differentiating neuronal tissue from non-neuronal tissue, identifying and spatially locating dysfunctional neuronal tissue, identifying, locating and monitoring intracranial trauma and central nervous system conditions, such as neurological disorders, and identifying and monitoring spinal cord, central and peripheral nerve function and abnormalities.

### BACKGROUND OF THE INVENTION

Methods and systems for assessing blood characteristics, blood flow, blood accumulation and deficiencies, and the location and condition of blood vessels, and for diagnosing conditions characterized by abnormalities in such properties, such as various cancers, are generally time consuming and invasive. Many of the diagnostic techniques currently available are inconvenient, painful, and invasive. Most surgical techniques damage blood vessels and many surgeries require extensive blood flow management to reduce blood loss during surgery and then to restore normal blood flow. In the operating room, intraoperative ultrasound and stereotaxic systems provide information about the location of various tissues, organs, blood vessels, tumors, etc. Ultrasound shows the location of some anatomical landmarks from the surface but, once surgery begins, ultrasound techniques do not provide information sufficient to locate blood vessels or identify areas of abnormal (excess or diminished) blood flow, or the like. Stereotaxic systems coupled with advanced imaging techniques have, at select hospitals, provided localization of physiological landmarks based upon the preoperative CT or MRI scans. However, studies have shown that the location of the physiological landmarks and important blood vessels, changes, particularly during invasive surgeries.

Thus, medical professionals often do not have reliable information concerning the location and characteristics of blood flow and blood conditions and parameters. Surgeons, for example, must make difficult decisions to reduce or eliminate blood flow to certain areas, thus potentially damaging tissue in these areas. Furthermore, surgeons must restore blood flow and repair blood vessels to ensure the recovery of the patient.

Methods and systems for identifying abnormal or pathological cells and tissue, particularly cancer, and for diagnosing cancerous conditions, are generally time consuming and invasive. Furthermore, many of the screening techniques currently available provide limited sensitivity and specificity. Tissue biopsies or samples may be taken, fixed and examined using various histological techniques. Since these diagnostic procedures are both invasive and expensive, and they are very stressful for patients undergoing testing, it is desirable to screen areas of suspected abnormalities first, to eliminate unnecessary trauma and expense. Diagnostic screening techniques used for detecting breast cancer, uterine and cervical cancers, colon and colo-rectal cancers, esophageal cancer and skin cancers are generally inadequate and unreliable. It is thus a high priority to develop methods and systems providing reliable, non-invasive screening techniques for identifying cancer cells that have a high degree of sensitivity and specificity.

The diagnostic value of performing a biopsy is dependent upon the selection of tissue for sampling. Many pathologies are not uniformly distributed and, therefore, the selection of tissue for sampling may be determinative of the diagnostic outcome. Additionally, unnecessary removal of tissue may result in localized trauma and, in some cases, may result in diminished function. Taking tissue samples from lymph nodes, for example, is essential to assess the progression of many cancers. Yet, removal of too much tissue, or removal of normal localized tissue having a specialized function may result in diminished function. It is therefore essential to identify and sample tissue that has the highest likelihood of including pathological cells, while avoiding the removal of healthy tissue.

A primary means for treatment of pathologies, such as cancer, is surgical removal. Many studies have shown that the clinical outcome is improved when more of the total amount of tumor tissue is removed. For gross total resections of tumors, the five year survival rate is significantly increased compared to subtotal resection. Both duration of survival and independent status of the patient are prolonged when the extent of resection is maximized in cancerous tissue. Current intraoperative techniques do not provide rapid differentiation of tumor tissue from normal surrounding tissue, however, particularly after resection of the tumor begins. Development of techniques that enhance the ability to identify tumor tissue intraoperatively may result in maximizing the degree of tumor resection, thereby prolonging survival.

Most current tumor detection techniques are performed prior to surgery to provide information about tumor location. Pre-surgical imaging methods include magnetic resonance imaging (MRI) and computerized tomography (CT). In the operating room, intraoperative ultrasound and stereotaxic systems provide information about the location of tumors. Ultrasound shows the location of the tumor from the surface but, once surgery begins, ultrasound techniques do not provide information sufficient to prevent the destruction of important functional tissue while permitting maximal removal of tumor tissue. Stereotaxic systems coupled with advanced imaging techniques have, at select hospitals, provided localization of tumor margins based upon the preoperative CT or MRI scans. However, studies have shown that the location of the tumor changes, particularly during invasive surgeries, and the actual tumor may extend 2-3 cm beyond where the image enhanced putative tumor is located on preoperative images.

One method currently available for determining the location of tumors is to obtain multiple biopsies during surgery and wait for results of microscopic examination of sections. This technique, known as multiple histological margin sampling, suffers several drawbacks. First, this is a time-consuming procedure and can add about 30 to 90 minutes (depending upon the number of samples taken) to the length of time the patient is under anesthesia. The increased time required for margin sampling leads to increased medical costs, as operating room time costs are high. Moreover, increased operating room time for the patient increases the probability of infection and complications arising from the anesthesia. Multiple histological margin sampling is prone to errors, as the pathologist must prepare and evaluate samples in short order. In addition, margin sampling does not truly evaluate all regions surrounding a primary tumor and some areas of residual tumor can be missed due to sampling error.

Thus, although patient outcome is dependent upon aggressive removal of tumor tissue, a surgeon often does not have reliable intraoperative information concerning the location and extent of the tumor. Surgeons must make difficult decisions between aggressively removing tissue and destroying surrounding functional tissue, and they may not know the true outcome of the procedure until permanent tissue sections are available about one week later. Additional surgical procedures may be required following examination of the histological studies.

Other techniques developed to improve imaging of solid tumor masses during surgery include determining the shape of visible luminescence spectra from normal and cancerous tissue. U.S. Pat. No. 4,930,516 teaches that the shape of visible luminescence spectra from normal and cancerous tissue are different. Specifically, there is a shift to blue with different luminescent intensity peaks in cancerous tissue as compared to normal tissue. Thus it is possible to distinguish cancerous tissue by exciting the tissue with a beam of ultraviolet (UV) light and comparing visible native luminescence emitted from the tissue with luminescence from a non-cancerous control of the same tissue type. Such a procedure is fraught with difficulties since a real time, spatial map of the tumor location is not provided for the use of a surgeon. Moreover, the use of UV light at an excitation wavelength can cause photodynamic changes to normal cells and is dangerous for use in an operating room. In addition, UV light penetrates only superficially into tissue and requires quartz optical components instead of glass.

Following the identification and localization of malignant tissue, or following surgical removal of malignant tissue, it is important to monitor the tissue in the area of malignancy for the reappearance or spreading of malignant tissue. Similarly, monitoring an area of interest such as malignant tissue during and/or following treatment with drugs, radiation therapy, or the like, is necessary to assess the efficacy of the treatment and to monitor the progression or recession of the malignancy. Convenient, inexpensive and minimally invasive techniques are desirable for performing these monitoring functions, and few effective systems are available.

Many experimental techniques have been applied to study the physiology of the nervous system. Several of those techniques are described below. Few diagnostic modalities provide reliable and cost effective screening for conditions manifesting cortical and intracranial abnormalities, such as head trauma, subdural hematomas, Alzheimer's disease, multiple sclerosis, stroke, ischemia, hypoxia, psychiatric conditions, and the like. Similarly, monitoring the progression of injuries and disease states manifesting cortical abnormalities generally requires the use of imaging modalities, such as magnetic resonance imaging (MRI), positron emission tomography (PET), computerized tomography (CT) scans, or the like, involving extensive and expensive equipment. Blockages and dysfunction of the spinal cord and central and peripheral nerves, as well as healing and regeneration of nerve tissue, are likewise difficult to diagnose and monitor. It would be very useful to provide an inexpensive device that could be used in doctors' offices, ambulances, clinics, and the like, to provide screening of patients to detect and monitor cortical and intracranial abnormalities.

Mapping of functional cortical activity is also important. Hill and Keynes observed that the nerve from the walking leg of the shore crab (Carcinus maenas) normally has a whitish opacity caused by light scattering, and that opacity changes evoked by electrical stimulation of that nerve were measurable. Hill, D. K. and Keynes, R. D., “Opacity Changes in Stimulated Nerve,” J. Physiol. 108:278-281 (1949). Since the publication of those results, experiments designed to learn more about the physiological mechanisms underlying the correlation between optical and electrical properties of neuronal tissue and to develop improved techniques for detecting and recording activity-evoked optical changes have been ongoing.

Several types of phenomenon relating to physiological neuronal activity have been detected. Thermographic studies have detected thermal radiation changes that take place during neuronal activation using infrared imaging techniques. Spectrophotometric techniques have been used to detect changes in absorption of the oxidizable fraction of cytochrome oxidase in brain tissue. Spectroscopic techniques such as electron microscopy and x-ray diffraction are not well-suited to studying physiological activity in living neuronal tissue because of the high risk of tissue damage.

Optical techniques have been developed and used for numerous applications. Light scattering has been used in the past to provide measurements of osmotic water permeability in suspensions of osmotically responsive vesicles and small c ells. (Verkman A S, “Optical methods to measure membrane transport processes,” J. Membrane Biol. 148:99-110, 1995.) Another study reported a method for the optical measurement of osmotic water transport in cultured cells. (Echevarria M, Verkman A S, “Optical measurement of osmotic water transport in cultured cells: role of glucose transporters,” J. Gen. Physiol. 99:573-589, 1992.)

Many biomolecules fluoresce as a result of excitation with emr at the wavelength of the molecule's absorption band. This excitation causes the molecule to emit part of the absorbed energy at a different wavelength, and the emission can be detected using fluorometric techniques. Most physiological studies measuring intrinsic fluorescence have selected for NADH, which is an important intermediate in oxidative catabolism. Furthermore, NADH concentration in neuronal tissue is believed to be correlated with neuronal activity. Upon excitation with ultraviolet light, NADH fluoresces at about 460 nm. Unfortunately, this technique would not be suitable for monitoring neuronal activity in humans, because illumination of in vivo neuronal tissue in vivo with ultraviolet light may cause serious tissue damage.

Another technique for detecting neuronal activity involves administration of a voltage-sensitive dye, whose optical properties change during changes in electrical activity of neuronal cells. The spatial resolution achieved by this technique is near the single cell level. For example, researchers have used the voltage-sensitive dye merocyanine oxazolone to map cortical function in a monkey model. Blasdel, G. G. and Salama, G., “Voltage Sensitive Dyes Reveal a Modular Organization Monkey Striate Cortex,” Nature 321:579-585, 1986. However, the use of these kinds of dyes would pose too great a risk for use in humans in view of their toxicity. Furthermore, such dyes are bleached by light and must be infused frequently.

Intrinsic changes in optical properties of cortical tissue have been assessed by reflection measurements of tissue in response to electrical or metabolic activity. Grinvald, A., et al., “Functional Architecture of Cortex Revealed by Optical Imaging of Intrinsic Signals,” Nature 324:361-364, 1986; Grinvald, A., et al., “Optical Imaging of Neuronal Activity, Physiological Reviews, 68:4, October 1988. Grinvald and his colleagues reported that some slow signals from hippocampus slices could be imaged using a CCD camera without signal averaging.

A CCD camera was used to detect intrinsic signals in a monkey model. Ts'o, D. Y., et al., “Functional Organization of Primate Visual Cortex Revealed by High Resolution Optical Imaging,” Science 249:417-420, 1990. The technique employed by Ts'o et al. would not be practical for human clinical use, since imaging of intrinsic signals was achieved by implanting a stainless steel optical chamber in the skull of a monkey and contacting the cortical tissue with an optical oil. Furthermore, in order to achieve sufficient signal to noise ratios, Ts'o e t al. had to average images over periods of time greater than 30 minutes per image.

The mechanisms responsible for intrinsic signals are not well understood. Possible sources of intrinsic signals include dilation of small blood vessels, neuronal activity-dependent release of potassium, and swelling of neurons and/or glial cells caused, for example, by ion fluxes or osmotic activity. Light having a wavelength in the range of 500 to 700 nm may also be reflected differently between active and quiescent tissue due to increased blood flow into regions of higher neuronal activity. Yet another factor which may contribute to intrinsic signals is a change in the ratio of oxyhemoglobin and deoxyhemoglobin in blood.

U.S. Pat. No. 5,215,095 discloses methods and apparatus for real time imaging of functional activity in cortical areas of a mammalian brain using intrinsic signals. A cortical area is illuminated, light reflected from the cortical area is detected, and digitized images of detected light are acquired and analyzed by subtractively combining at least two image frames to provide a difference image.

U.S. Pat. No. 5,438,989 discloses a method for imaging margins, grade and dimensions of solid tumor tissue by illuminating the area of interest with high intensity electromagnetic radiation containing a wavelength absorbed by a contrast agent, obtaining a background video image of the area of interest, administering a contrast agent, and obtaining subsequent video images that, when compared with the background image, identify the solid tumor tissue as an area of changed absorption. U.S. Pat. No. 5,699,798 discloses methods and apparatus for optically distinguishing between tumor and non-tumor tissue, and imaging margins and dimensions of tumors during surgical or diagnostic procedures.

U.S. Pat. No. 5,465,718 discloses a method for imaging tumor tissue adjacent to nerve tissue to aid in selective resection of tumor tissue using stimulation of a nerve with an appropriate paradigm to activate the nerve, permitting imaging of the active nerve. The '718 patent also discloses methods for imaging of cortical functional areas and dysfunctional areas, methods for visualizing intrinsic signals, and methods for enhancing the sensitivity and contrast of images. U.S. Pat. No. 5,845,639 discloses optical imaging methods and apparatus for detecting differences in blood flow rates and flow changes, as well as cortical areas of neuronal inhibition.

A need in the art remains for methods and apparatus for detecting physiological properties in an area of interest that are accurate, reliable, conveniently implemented, cost effective and non-traumatic.

### SUMMARY OF THE INVENTION

The methods and systems described herein may be used to detect various physiological properties and characteristics, such as, but not limited to, abnormal tissues, such as malignant tissues, and abnormalities of the circulatory and nervous systems. Methods and systems of the present invention distinguish between normal and abnormal blood characteristics and blood flow using optical spectroscopic detection techniques and, optionally, contrast enhancing agents, and aid in identifying blood and blood vessel abnormalities for surgical, diagnostic, and monitoring applications. For example, optical detection techniques of the present invention may be used in diagnostic screening applications or may be used by a surgeon intraoperatively, to distinguish between normal and abnormal blood characteristics and blood flow, and to identify blood vessels, with a high degree of spatial resolution. Methods and systems of the present invention may also be used to monitor the progression or recession of a disease or condition characterized by abnormal blood characteristics, blood flow or vascularization, and to monitor the efficacy of treatment protocols or agents. The optical detection techniques of the present invention provide information and results in “real-time,” and thus may be interfaced with stereotaxic systems used during surgical procedures to accurately locate areas of abnormal blood flow or restriction during surgeries. Typically, the illuminating or detecting is performed external to a blood vessel being studied by one optical source or at least one detector located external to the blood vessel. In some embodiments, an optical source or an array of optical sources and an optical detectors or an array of optical detectors are employed, wherein the optical detector(s) is separately located from the optical sources.

Many physiological changes and conditions are characterized by changes in the vascularization or blood flow in tissue. It is recognized, for example, that tumors and cancerous tissue possess abnormal patterns of vascularization and blood flow compared to normal, non-tumor tissue. See, for example, Dewhirst Mark W, “Angiogenesis and blood flow in solid tumors,” in Teicher, Beverly A, ed., Drug Resistance in Oncology, Marcel Dekker, Inc.: New York, 1993. Abnormal tissue, such as tumor tissue, also frequently exhibits various pathophysiological features, and the characteristics of blood in abnormal tissue thus are frequently different from the characteristics of blood in normal, functional tissue. Methods and systems of the present invention, detecting optical properties of an area of interest, are capable, for example, of differentiating oxygenated from de-oxygenated blood and, at highly sensitive levels, may differentiate the oxygen content of blood, as well as other blood characteristics. Similarly, tissue of the central and peripheral nervous systems may exhibit different blood characteristics and different blood flow during periods of activation and periods of quiescence. Changes in central and peripheral nervous system tissue produced by various conditions, particularly neurological conditions manifesting changes in the character of cortical tissue, such as beta amyloid plaques, demyelination, and the like, are also evidenced by changes in blood characteristics and/or blood flow in such tissue.

Methods and systems of the present invention may also be used to map blood flow in areas of interest and to detect blood flow abnormalities, such as blood clots or hematomas. Optical methods and systems of the present invention are therefore useful for diagnosing and monitoring conditions manifesting intracranial abnormalities, such as head trauma, subdural hematomas, stroke, ischemia, hypoxia, epilepsy, and the like, as well as heart and blood vessel conditions, including clots and hematomas, characterized by abnormal blood characteristics and abnormal blood flow.

The methods and systems described herein also distinguish between normal and abnormal, or pathological tissue, such as cancerous tissue, using optical detection techniques and contrast enhancing agents, and aid in identifying pathological tissue during surgical, diagnostic, monitoring and biopsy procedures. For example, optical detection techniques of the present invention may be used in diagnostic screening applications to identify pathological tissue, such as cancerous tissue. In addition, the methods and apparatus of the present invention are used to identify margins and dimensions of pathological tissue during surgical procedures, and to grade and characterize pathological tissue, particularly cancerous tissue. Additionally, methods and systems of the present invention may be used as a biopsy aid to identify potentially abnormal tissue that should be included in a biopsy sample; for monitoring the progression or recession of a pathological condition, such as cancer; and/or for monitoring the efficacy of treatment agents or protocols. The optical detection techniques of the present invention provide information and results in “real-time” and with a high degree of spatial resolution, and thus may be used intraoperatively or be interfaced with stereotaxic systems used during surgical procedures to accurately locate the malignant tissue during surgeries.

The methods and systems described may further be used to identify areas of neuronal activity and dysfunction with a high degree of spatial resolution during surgical or diagnostic procedures, and to monitor neuronal activity to assess tissue viability, function, recovery, degeneration and the like. For example, optical detection techniques of the present invention can be used by a surgeon intraoperatively to distinguish between functional and dysfunctional neuronal tissue, and to distinguish between neuronal tissue and surrounding non-neuronal tissue. In addition, the methods and apparatus of the present invention can be used to identify, and locate, with a high degree of spatial resolution, neuronal tissue dedicated to important functional activities such as vision, movement, sensation, memory and language. Similarly, the methods and apparatus of the present invention can be used to detect areas of “abnormal” neuronal activity, whether that neuronal activity is unusually “high” or “low,” such as epileptic foci (“high”) or non-viable or dysfunctional neuronal tissue (“low”). Methods and systems of the present invention can also be used to identify and locate individual nerves and identify areas of nerve damage or dysfunction.

Optical methods and systems of the present invention are also useful for diagnosing and monitoring conditions manifesting cortical and intracranial abnormalities, such as head trauma, subdural hematomas, Alzheimer's disease, Parkinson's disease, ALS, multiple sclerosis, stroke, ischemia, hypoxia, psychiatric conditions, epilepsy, migraine, spreading depression, as well as psychiatric disorders such as depression, anxiety, bipolar disorder, schizophrenia, and the like. The methods and systems described herein distinguish between normal, functional cortex and dysfunctional cortex based upon one or more intrinsic or extrinsic optical properties and are therefore useful for identifying margins and dimensions of nonfunctional tissue, as well as identifying areas of functional tissue during surgical, diagnostic, and biopsy procedures. The optical techniques may be used as a biopsy aid to identify potentially abnormal tissue that should be included in a biopsy sample; for monitoring the progression or recession of abnormal or dysfunctional tissue, particularly abnormal or dysfunctional cortical tissue; and/or for monitoring the efficacy of treatment protocols and agents, particularly treatment protocols and agents intended to improve cortical, intracranial, or peripheral nervous system abnormalities. The optical detection techniques of the present invention provide information and results in “real-time,” and thus may be used intraoperatively or be interfaced with stereotaxic systems used during surgical procedures to accurately locate the malignant tissue during surgeries.

Detection and monitoring of intracranial traumas, such as subdural hemotomas, ischemia, hypoxia, loss of cortical function, and the like, are important applications for methods and systems of the present invention. There is an acute need for non-invasive or semi-invasive tools for diagnosing and monitoring such intracranial conditions for use in critical care circumstances, such as in ambulances, emergency rooms, clinics, and the like. Implementing techniques of the present invention, optical source and detector pairs, or arrays of multiple optical sources and detectors may be mounted on a patient's scalp for illumination and detection, through the cranium, of the underlying tissue and cortical surface. An array of optical sources and detectors may be arranged, for example, in a flexible pad that conforms to the surface contours of an area of interest, such as the skull. Alternatively, optical sources and detectors may be placed intracranially to provide direct illumination and detection of optical and/or geometrical properties of the area of interest. According to one embodiment, one or more optical source/detector pairs, or one or more arrays of optical sources and detectors, may be provided in a sterile package and placed intracranially. The optical sources and detectors are preferably maintained in a generally fixed relationship to the area of interest during acquisition of data relating to optical and/or geometrical properties of the area of interest. Panels of control data may be provided from empirically derived studies of various types of conditions or tissues, or may be derived from a control area of interest of the patient under study that is known or suspected to contain normal tissue.

Many conditions and disease states affecting the central nervous system produce cortical abnormalities that are detectable and may be monitored using the methods and systems of the present invention. Alzheimer's disease, for example, produces beta amyloid plaques that are detectable and may be spatially located using the optical techniques of the present invention. Demyelination resulting from multiple sclerosis is likewise detectable and may be spatially located using the optical techniques of the present invention. Migraine headaches, spreading depression, ALS, Parkinson's and other neurogenerative disorders, as well as psychiatric disorders such as depression, anxiety, bipolar disorder, and schizophrenia, manifest characteristic cortical abnormalities that are detectable and may be spatially located using the optical techniques of the present invention. Yet another application for methods and systems of the present invention involves in situ monitoring of an area of interest to evaluate the progression, or recession, of a condition involving abnormal tissue, such as cortical abnormalities, in an area of interest, and to monitor, in situ, the effect of a treatment regimen or agent on an identified or suspected area of interest, such as cortex. Methods and systems of the present invention may be employed, for example, to provide frequent screening or monitoring of cortical tissue to rapidly detect any disease progression that would benefit from additional or differential treatment agents or regimen. Screening and monitoring may also be implemented to evaluate the need for additional testing using more expensive and less accessible techniques, such as MRI.

Methods and systems of the present invention may also provide diagnostic screening and monitoring of the spinal cord and central and peripheral nerves. Because active nerves have distinct optical characteristics during periods of activation, functional nerve tissue is distinguishable from dysfunctional or pathological nerve tissue using the methods and systems of the present invention. Severed or damaged nerves, as well as sites of nerve damage, may be spatially located using optical techniques. Similarly, healing or regeneration of nerves may be monitored. According to one embodiment, an array of optical sources and detectors is positioned in proximity to an area of interest including a nerve path. Data corresponding to one or more optical properties of tissue in the area of interest is acquired during stimulation of the nerve. Activated nerve tissue is distinguishable from non-functional nerve tissue and surrounding tissue based on one more optical properties. Sites of nerve blockage, dysfunction, pathology, and the like, may thus be identified with a high degree of spatial resolution. Control data may be empirically derived, or may be acquired from a corresponding area of interest known or believed to contain corresponding, functional nerve tissue.

The present invention thus contemplates screening devices for detecting and locating spinal cord, central and peripheral nerve damage for use in ambulances, emergency rooms, clinics, and the like. Such devices may be used as diagnostic and monitoring systems for spinal cord function, retinal function, peripheral nerve function, including the diagnosis and monitoring of carpal tunnel syndrome, and for numerous other applications.

Methods and systems of the present invention may also be used to identify physiological conditions associated with and to evaluate test agents and conditions for diagnosis and treatment of various disorders, and pathological conditions, including migraine headaches, spreading depression, epilepsy, Alzheimer's disease, multiple sclerosis, toxicities affecting neuronal tissue such as ethanol toxicity, psychiatric disorders such as depression, anxiety, bipolar disorder, schizophrenia, Parkinson's disease, ALS, and other neurodegenerative disorders, inflammation, infection, trauma, malignancies, angiogenesis, wound healing, immune deficiencies, and the like. Techniques and systems of the present invention for identifying and spatially locating abnormal intracranial and cortical conditions are useful for diagnosis of many conditions, and particularly useful as non-invasive or semi-invasive techniques for screening areas of interest. Test agents and conditions may also be tested for safety and efficacy for applications such as toxicology, learning and memory, bone growth and maintenance, muscle and blood systems, sensory-input systems, and the like. The progression of such disorders and physiological conditions may also be monitored using the methods and systems of the present invention. Additionally, methods and systems of the present invention may be used intraoperatively, or interfaced with stereotaxic systems to assist medical personnel in spatially locating areas of dysfunctional or diseased or non-viable tissue during surgery. The methods and systems of the present invention may be used with or without contrast enhancing agents. The use of contrast-enhancing agents for certain applications provides data having high sensitivity and specificity. Administration of contrast enhancing agents may, for example, change optical absorption properties, optical scattering properties, birefringence, or the like. Alternatively or additionally, contrast enhancing agents may exhibit different dynamics, such as different perfusion rates, clearance rates, or the like, in normal and abnormal tissue. Such differences result, in many cases, from abnormal or different vascularization in such tissues. For some applications, it may be desirable to employ multiple contrast enhancing agents, each agent having different spectral properties. The contrast enhancing agents are non-toxic to normal cells and do not interfere with normal metabolic activities at the area of interest. In one exemplary embodiment, the dynamics of the perfusion of a contrast enhancement agent administered in the blood through normal tissue differ from the dynamics of dye perfusion through abnormal tissue, such as, but not limited to, cancerous tissue.

Examples of contrast enhancing agents include fluorescent and phosphorescent materials, photodynamic dyes, indocyanines, fluoresceins, hematoporphyrins, and fluoresdamines, agents that are used topically, such as iodine, weak acidic and basic agents, and the like. Indocyanine green, which has a broad absorption wavelength range and a peak absorption in the range of 730 nm to 840 nm, is a suitable contrast enhancing agent for detection of cancerous tissue in diagnostic and intraoperative procedures. Iodine and weak acidic and basic agents are suitable contrast enhancing agents for topical application and screening for cancerous tissue on the surface of tissue, such as cervical tissue, colo-rectal tissue, intestinal system tissue, and the like. Agents that preferentially sequester in abnormal or pathological tissue may be used. The contrast enhancing agent may be administered intravenously, intraarterially, subcutaneously, topically, or using any route of administration that delivers the agent to the area of interest. Detectors appropriate for use with the contrast enhancing agents employed with methods and systems of the present invention are well known in the art. The systems of the present invention employ one or more electromagnetic radiation (emr) optical source(s) for illuminating an area of interest (i.e., an area to be screened or an area believed to contain abnormal or pathological tissue), and one or more optical detector(s) capable of detecting and acquiring data relating to one or more optical properties of the area of interest. The optical source(s) and detector(s) may be selected and located to acquire data relating to optical properties of an area of interest that is exposed, or that underlies skin, tissue, bone, dura, or the like. Epi-illumination and reflective detection are preferred for many applications. For some applications, transillumination techniques are used, optionally following administration of a contrast enhancing agent, to identify abnormalities within a tissue sample in situ.

The optical detector(s) may be used to acquire data for analysis in a static mode, or multiple data sets may be acquired at various time intervals for comparison in a dynamic mode. The optical detector(s) may, for example, acquire control data representing the “normal” or “background” optical properties of an area of interest, and then acquire subsequent data representing the optical properties of an area of interest following administration of a stimulus and/or a contrast-enhancing agent, or during a monitoring interval. The subsequent data is compared to the control data, or to empirically determined standards, to identify changes in optical properties of corresponding spatial locations in the data set that are representative of normal and abnormal blood characteristics, blood flow rates, blood vessels and/or normal and abnormal tissue and/or representative of activity or dysfunction.

Optical source(s) may provide continuous or non-continuous illumination. Various types of optical detectors may be used, depending on the emr source(s) used, the optical property being detected, the type of data being collected, certain properties of the area of interest, the desired data processing operations, the format in which the data is displayed, and the type of application, e.g., intraoperative, diagnostic, biopsy, monitoring, or the like. For some applications, emr sources providing continuous, uniform illumination are preferred, while non-continuous illumination using time domain or frequency domain illumination sources are preferred for some applications.

Changes in optical properties that may be indicative of changes in blood characteristics, blood flow, blood vessels, abnormal tissues, as well as indicative of activity or dysfunction in central and peripheral nervous system tissue include, for example, reflection, refraction, diffraction, absorption, scattering, birefringence, refractive index, Kerr effect, and the like. The optical source and detection system may be incorporated in an apparatus for use external to the area of interest, or optical detection components may be mounted in an invasive or semi-invasive system, such as an endoscope, laparoscope, biopsy device or probe, or may be provided as individual optical fibers or bundles of optical fibers, or the like.

Numerous devices for acquiring, processing and displaying data representative of one or more optical properties of spatially localized and identified areas in an area of interest can be employed. In general, any type of photon detector may be utilized as an optical detector. The optical detector generally includes photon sensitive elements and optical elements that enhance or process the detected optical signals, such as lenses, polarizers, objectives, and the like. In a simple form, the apparatus of the present invention may include one or more optical fibers operably connected to one or more emr sources that illuminates tissue, with corresponding optical fibers operably connected to an optical detector, such as a photodiode, that detects one or more optical properties of the illuminated tissue. According to another embodiment, a video camera acquires control and subsequent images of an area of interest that can then be compared to identify abnormal blood characteristics, blood flow rates, or blood vessels. According to another embodiment, areas of abnormal tissues may be identified. In yet another embodiment, control and subsequent images of an area of interest may me compared to identify areas of active and/or dysfunctional nervous tissue. Examination of such data provides precise spatial location of abnormalities, such as cancer, blood clots, hematomas, or central or peripheral nervous system activity. Apparatus suitable for obtaining data relating to one or more optical properties of an area of interest have been described in the patents incorporated herein by reference and are more fully described below.

For most surgical, diagnostic, and monitoring uses, the optical detector preferably provides data having a high degree of spatial resolution at a magnification sufficient to precisely locate the areas of abnormality of blood characteristics, blood flow, or blood vessels. Several data sets are preferably acquired over a predetermined time period and combined, such as by averaging, to provide data sets for analysis and comparison. Methods and systems of the present invention may be used in a static mode that provides a comparison of optical properties of different spatial locations in an area of interest, to spatially locate areas showing differential optical properties and thereby locate areas of differential blood characteristics, blood flow, and/or blood vessels, as well as abnormal tissues, and/or active and dysfunctional, or abnormal, tissues of the central or peripheral nervous system. A comparison of optical properties of two different areas of tissue may also be made in a static mode. Thus, the optical properties of an area of tissue believed to contain abnormal tissue may be compared to the optical properties of another area of the same type of tissue believed to contain normal tissue. In this embodiment, the presumed normal area of interest serves as the control, or background data set for comparison with the area of interest believed to contain one or more abnormalities.

Operation of methods and systems of the present invention in a dynamic mode compares data acquired from corresponding spatial locations at various time points. While it is preferred, for many applications, to acquire control data sets from the area of interest of each patient prior to administration of the contrast enhancing agent to compare with subsequent data sets acquired from the same area of interest in the same patient subsequent to administration of the contrast enhancing agent, it is also possible to compare data sets to empirically determined standard or control data sets. Diagnostic screening may be performed by comparing the patient data set to standard data sets representative of cortical optical properties indicative of various disease states or conditions. Methods and systems of the present invention may also be used as a biopsy aid to spatially locate areas within an area of interest having a high likelihood of being abnormal or dysfunctional.

Various data processing techniques may be advantageously used to assess the data collected in accordance with the present invention. Data may be analyzed and displayed in a variety of formats. Processing may include averaging or otherwise combining a plurality of data sets to produce control, subsequent or comparison data sets. Other optical data processing techniques include frequency domain methods such as Fourier or wavelet transformations of the optical data, or spatial domain methods such as convolutions, geometrical transformations, data differencing, and the like. According to one embodiment, statistically significant changes in intrinsic or extrinsic optical properties of central and peripheral nervous system tissue may be determined for various types of tissue activity, contrast enhancing agents, and the like. Comparison of a data set acquired following stimulation of central or peripheral nervous system activity to a control data set representing statistically significant optical changes provides identification of spatial locations within an area of interest evidencing statistically significant changes indicative of normal activity, dysfunction, or the like.

Data processing may also include amplification of certain signals or portions of a data set (e.g., areas of an image) to enhance the contrast seen in data set comparisons, and to thereby identify areas of abnormal tissue with a high degree of spatial resolution. For example, according to one embodiment, images are processed using a transformation in which data point brightness values are remapped to cover a broader dynamic range of values. A “low” value may be selected and mapped to zero, with all data point brightness values at or below the low value set to zero, and a “high” value may be selected and mapped to a selected value, with all data point brightness values at or above the high value mapped to the high value. Data having an intermediate brightness value, representing the dynamic changes in brightness indicative of changes in optical properties, may be mapped to linearly or logarithmically increasing brightness values. This type of processing manipulation is frequently referred to as a “histogram stretch” or point transformation, and can be used according to the present invention to enhance the contrast of data sets, such as images, representing differences in tissue type.

Data processing techniques may also be used to manipulate data sets to provide more accurate combined and comparison data. For example, patient movement, respiration, heartbeat or reflex activity may shift an area of interest during detection of optical properties and data collection. It is important that corresponding data points in data sets (such as corresponding pixels of an image) are precisely aligned, spatially, to provide accurate combined and comparison data. Such alignment may be accomplished manually by a practitioner having specialized skill and expertise, or using a variety of mechanical and/or mathematical means. Emr source(s) and optical detector(s) may, for example, be mounted in a relatively “fixed” condition in proximity to an area of interest. Optical markers may be fixed at an area of interest and detected as the data is collected to aid in manual alignment or mathematical manipulation. Motion artifacts may be reduced or substantially eliminated by timing the acquisition of data to the cycle of respiration, heartbeat, or the like, to normalize the data. Various processing techniques are described below and in the patents incorporated herein by reference.

Comparison data may be displayed in a variety of ways. For example, comparison data may be displayed in a graphical format that highlights optical differences differentiating normal from abnormal tissue. A preferred technique for presenting and displaying comparison data is in the form of visual images, or photographic frames, corresponding to the area of interest. This format provides a visualizable spatial location (two- or three-dimensional) of an area of interest that is useful for treatment, diagnosis and monitoring.

To enhance and provide better visualization of optical contrast between abnormal and normal tissue, comparison images may be processed to provide an enhanced contrast grey scale or even a color image. A look up table (“LUT”) may be provided, for example, that converts the grey scale values for each pixel to a different (higher contrast) grey scale value, or to a color value. Color values may map to a range of grey scale values, or color may be used to distinguish between positive-going and negative-going optical changes. In general, color-converted images provide higher contrast images that highlight changes in optical properties representing areas demonstrating different blood characteristics, blood flow, or blood vessel abnormalities, as well as abnormal tissues and active and dysfunctional, or abnormal, tissues of the central or peripheral nervous system.

In operation, an area of interest in a patient is illuminated with electromagnetic radiation (emr) while one or a series of data points or data sets representing one or more optical properties of spatially definable areas in the area of interest is acquired. Data sets are acquired before and/or after the optional administration of a contrast enhancing agent. The area of interest may be exposed to the emr source(s), or may underlie skin, tissue, bone, dura, or the like, provided that the emr source(s) is selected and positioned to penetrate tissue overlying the area of interest. Alternatively, the area of interest may be located within tissue, and the emr source(s) and detector(s) selected and positioned for transillumination of the area of interest.

For operation in a static mode, using a contrast enhancing agent, the contrast enhancing agent is administered, such as by injection or topical application, to an area of interest, and a data set mapping one or more optical properties to spatial locations in the area of interest is acquired. Spatial locations evidencing contrasting optical properties representative of areas of different blood characteristics, blood flow, blood volume, or blood vessel properties highlight areas of “normal” and “abnormal” properties. Application of a topical contrast enhancing agent such as iodine or a weak acidic or basic agent to the surface of an area of interest, such as cervical tissue, colo-rectal tissue, digestive system tissue, esophageal tissue, or the like, for example, is followed by illumination of the area of interest and detection of differential optical properties at different spatial locations within the area of interest. Similarly, injections of a contrast enhancing agent, such as indocyanine green, followed by illumination of the area of interest and detection of differential optical properties corresponding to different spatial locations within the area of interest, provides differentiation and spatial localization of abnormal tissue evidencing, for example, blood flow abnormalities, such as cancerous tissue, from surrounding normal tissue.

For static mode operation for investigating central and peripheral nervous systems, a data set mapping one or more intrinsic optical properties to spatial locations in the area of interest is acquired. Spatial locations evidencing contrasting intrinsic optical properties highlight areas of normal, functional tissue and dysfunctional tissue, or active and inactive tissue. Active tissue, such as functional neuronal tissue, is mapped by stimulation of activity in an area of interest, such as cortex, followed by illumination of the area of interest and detection of differential optical properties at different spatial locations within the area of interest. Detection of differential optical properties corresponding to different spatial locations within the area of interest provides differentiation and spatial localization of active, functional tissue, such as cortical tissue having various functional roles, from surrounding tissue having a different function, or from dysfunctional surrounding tissue.

Additionally, operation in a static mode may involve illumination and acquisition of data sets from two spatially separated locations and comparison of the data sets at one or more time points. Thus, for example, data representative of the optical properties of two different areas of tissue may be acquired at predetermined time intervals following administration of a contrast enhancing agent, such as indocyanine green. One of the areas of interest is presumed to contain “normal” tissue. Data from the “normal” area of interest is compared to data from the other area of interest to detect and spatially localize differential optical properties that are indicative of differences in blood characteristics, blood flow, and/or blood vessels, as well as abnormal tissue. Similarly, data representative of the intrinsic optical properties of two different areas of cortical tissue may be acquired at predetermined time intervals following stimulation of activity. One of the areas of interest is presumed to contain “normal” tissue. Data from the “normal” area of interest is compared to data from the other area of interest to detect and spatially localize differential intrinsic optical properties that are indicative of active, functional tissue or, alternatively, dysfunctional tissue. Acquired data may be compared to control or background data during operation in a static or a dynamic mode. Control data may represent standards derived from optical properties of empirical data samples of desired tissue populations. Control data may thus be derived representing various normal tissue types as well as various abnormal tissue types, such as different types and grades of tumors. Comparison of data acquired from an area of interest to various types of control data may then provide identification and spatial localization of abnormal tissue, such as cancer, as well as typing of the abnormal tissue, such as identifying particular cancers, and grading of cancerous tissue. For abnormalities such as cancer, it may be desirable to employ a contrast enhancing agent, and to compare multiple data sets acquired at intervals following administration of a contrast enhancing agent to control data to observe changes in the optical properties of tissue at the area of interest at predetermined time intervals following administration of the contrast enhancing agent.

In a similar manner, data acquired during a resting, or quiescent phase, or data acquired during stimulation of activity, may be compared to control or background data during operation in a static or a dynamic mode. Control data may represent standards derived from optical properties of empirical data samples of desired tissue populations. Control data may thus be derived representing various normal or active, functional central and peripheral nerve tissue types as well as various dysfunctional tissue types indicative of intracranial and cortical abnormalities. Comparison of data acquired following stimulation of activity to various types of control data provides identification and spatial localization of functional, as well as dysfunctional tissue, and may provide characterization of the dysfunctional tissue, such as identifying conditions manifesting characteristic cortical abnormalities. It may be desirable to compare multiple data sets acquired at intervals following stimulation of activity to control data to observe changes in the intrinsic optical properties of tissue at the area of interest at predetermined time intervals following the stimulation of activity. According to one embodiment, statistically significant, contrast enhanced changes in optical properties of tissue may be determined empirically for various types of tissue, blood characteristics, blood flow, blood vessel morphologies, cancers, contrast enhancing agents, and the like. Comparison of a data set acquired following administration of a contrast enhancing agent to a control data set representing statistically significant changes provides identification of spatial locations within an area of interest evidencing statistically significant changes indicative of abnormalities.

In another dynamic mode, data acquired corresponding to an optical property of an area of interest prior to administration of a contrast enhancing agent represents control, or background, data. A series of data sets is preferably combined, for example by averaging, to obtain a control data set. The control data set is stored for comparison with data collected subsequently. Alternatively, control or background data corresponding to various conditions of tissue and areas of interest may be acquired, stored, and used for comparison. Control data sets may also be acquired, in real time, from an area of interest believed to contain normal tissue. A subsequent data set representing the corresponding optical property is acquired during a subsequent time period following administration of a contrast enhancing agent. A series of subsequent data sets is preferably combined, for example by averaging, to obtain a subsequent data set. Subsequent data sets are compared with one or more control data set(s) to obtain comparison data set(s), preferably difference data set(s). Comparison data sets are then examined for evidence of changes in optical properties representative of areas of abnormal versus normal tissue within an area of interest.

According to one embodiment, statistically significant, contrast enhanced changes in optical properties of tissue may be determined empirically for various types of tissue, blood abnormalities, cancers, contrast enhancing agents, and the like. Comparison of a data set acquired following administration of a contrast enhancing agent to a control data set representing statistically significant changes provides identification of spatial locations within an area of interest evidencing statistically significant changes indicative of abnormalities.

In yet another dynamic mode pertaining to detecting abnormalities of the central and peripheral nervous systems, data is acquired corresponding to intrinsic optical properties of spatial locations within an area of interest prior to stimulation of activity represents control, or background, data. A series of data sets is preferably combined, for example by averaging, to obtain a control data set. The control data set is stored for comparison with data collected subsequently. Alternatively, control or background data corresponding to various conditions of tissue and areas of interest may be acquired, stored, and used for comparison. Control data sets may also be acquired, in real time, from an area of interest believed to contain normal tissue. A subsequent data set representing the corresponding intrinsic optical properties of corresponding spatial locations within the area of interest is acquired during a subsequent time period following stimulation of activity. A series of subsequent data sets is preferably combined, for example by averaging, to obtain a subsequent data set. Subsequent data sets are compared with one or more control data set(s) to obtain comparison data set(s), preferably difference data set(s). Comparison data sets are then examined for evidence of changes in intrinsic optical properties representative of areas of functional versus inactive or non-functional tissue within an area of interest.

According to one embodiment, the methods and systems described, herein may be employed to obtain three-dimensional information of an area of interest suspected to contain abnormal tissue by: (a) illuminating the area of interest with a least two different wavelengths of emr; (b) obtaining a sequence of control data sets corresponding to each wavelength of emr; (c) administering a contrasting enhancing agent, or in the case of analyzing central and peripheral nervous systems, administering a stimulus producing central or peripheral nervous system activity and, optionally administering a contrasting enhancing agent; (d) obtaining a sequence of subsequent data sets for each wavelength of emr; (e) obtaining a series of comparison data sets for each wavelength of light by subtracting the control data set from the subsequent data set or alternatively, in the case of fluorescent dyes, subtracting the subsequent image from the control image; and (f) obtaining an enhanced comparison data set by ratioing the first comparison data set to the second comparison data set. Data corresponding to three dimensional spatial locations may also be acquired using multiple agents having different spectral properties, and by employing optical tomography techniques. Specifically, photon time-of-flight techniques and frequency domain methods may also be used.

Contrast enhancing agents suitable for use in the present invention enhance differences in the optical properties, or optical contrast, between cells and tissues having different properties. Administration of contrast enhancing agents may, for example, change optical absorption properties, optical scattering properties, birefringence, or the like, differentially in normal and abnormal cells. Alternatively or additionally, contrast enhancing agents may exhibit different dynamics, such as different perfusion rates, clearance rates, or the like, in different tissues, such as normal and abnormal tissue, or may sequester preferentially in abnormal tissue. For some applications, it may be desirable to employ multiple contrast enhancing agents, each agent having different spectral properties. The contrast enhancing agents are non-toxic to normal cells and do not interfere with normal metabolic activities at the area of interest.

Examples of contrast enhancing agents include fluorescent and phosphorescent materials, photodynamic dyes, indocyanines, fluoresceins, hematoporphyrins, and fluoresdamines, agents that are used topically, such as iodine, weak acidic and basic agents, and the like. The contrast enhancing agent may be administered intravenously, intraarterially, subcutaneously, topically, or using any route of administration that delivers the agent to the area of interest. Indocyanine green, which has a broad absorption wavelength range and a peak absorption in the range of 730 nm to 840 nm, is a suitable contrast enhancing agent for detection of cancerous tissue in diagnostic and intraoperative procedures. Iodine and weak acidic and basic agents are suitable contrast enhancing agents for topical application and screening for cancerous tissue on the surface of tissue, such as cervical tissue, colo-rectal tissue, intestinal system tissue, and the like. Agents that preferentially sequester in abnormal or pathological tissue may be used. Detectors appropriate for use with the contrast enhancing agents employed with methods and systems of the present invention are well known in the art.

The systems of the present invention employ one or more electromagnetic radiation (emr) optical source(s) for illuminating an area of interest (i.e., an area to be screened or an area believed to contain abnormal or pathological tissue), and one or more optical detector(s) capable of detecting and acquiring data relating to one or more optical properties of the area of interest. The optical source(s) and detector(s) may be selected and located to acquire data relating to optical properties of an area of interest that is exposed, or that underlies skin, tissue, bone, dura, or the like. Epi-illumination and reflective detection are preferred for many applications. For some applications, transillumination techniques are used, following administration of a contrast enhancing agent, to identify abnormalities within a tissue sample in situ, such as a breast.

The optical detector(s) may be used to acquire data for analysis in a static mode, or multiple data sets may be acquired at various time intervals for comparison in a dynamic mode. The optical detector(s) may, for example, acquire control data representing the “normal” or “background” optical properties of an area of interest, and then acquire subsequent data representing the optical properties of an area of interest following administration of a contrast-enhancing agent, or during a monitoring interval. The subsequent data is compared to the control data, or to empirically determined standards, to identify changes in optical properties of corresponding spatial locations in the data set that are representative of normal and abnormal tissue.

Optical source(s) may provide continuous or non-continuous illumination. Various types of optical detectors may be used, depending on the emr source(s) used, the optical property being detected, the type of data being collected, certain properties of the area of interest, the desired data processing operations, the format in which the data is displayed, and the type of application, e.g., intraoperative, diagnostic, biopsy, monitoring, or the like. For some applications, emr sources providing continuous, uniform illumination are preferred, while non-continuous illumination using time domain or frequency domain illumination sources are preferred for some applications.

Changes in optical properties that may be indicative of abnormalities include, for example, reflection, refraction, diffraction, absorption, scattering, birefringence, refractive index, Kerr effect, and the like. The optical source and detection system may be incorporated in an apparatus for use external to the area of interest, or optical detection components may be mounted in an invasive or semi-invasive system, such as an endoscope, laparoscope, biopsy device or probe, or may be provided as individual optical fibers or bundles of optical fibers, or the like.

Numerous devices for acquiring, processing and displaying data representative of one or more optical properties of spatially localized and identified areas in an area of interest can be employed. In general, any type of photon detector may be utilized as an optical detector. The optical detector generally includes photon sensitive elements and optical elements that enhance or process the detected optical signals, such as lenses, polarizers, objectives, and the like. In a simple form, the apparatus of the present invention may include one or more optical fibers operably connected to one or more emr sources that illuminates tissue, with corresponding optical fibers operably connected to an optical detector, such as a photodiode, that detects one or more optical properties of the illuminated tissue. According to another embodiment, a video camera acquires control and subsequent images of an area of interest that can then be compared to identify areas of abnormal tissue. Examination of such data elucidates the precise spatial location of tissue abnormalities and permits characterization of abnormal tissue, such as cancerous tissue. Apparatus and methods suitable for obtaining data relating to one or more optical properties of an area of interest have been described in the patents incorporated herein by reference and are more fully described below.

For most surgical, diagnostic, and monitoring uses, the optical detector preferably provides data having a high degree of spatial resolution at a magnification sufficient to precisely locate the margins of abnormal tissue, such as tumors and cancerous tissue. Several data sets are preferably acquired over a predetermined time period and combined, such as by averaging, to provide data sets for analysis and comparison. Methods and systems of the present invention may be used in a static mode that provides a comparison of optical properties of different spatial locations in an area of interest, to spatially locate areas showing differential contrast enhancement and thereby locate areas of normal and abnormal tissue. A comparison of optical properties of two different areas of interest may also be made in a static mode. Thus, following administration of a contrast enhancing agent, an area of interest believed to contain abnormal tissue may be compared to another area of interest of the same type of tissue believed to contain normal tissue. In this embodiment, the presumed normal area of interest provides the control, or background data set for comparison with the area of interest believed to contain abnormal tissue.

Operation of methods and systems of the present invention in a dynamic mode compares data acquired from corresponding spatial locations at various time points. While it is preferred, for many applications, to acquire control data sets from the area of interest of each patient prior to administration of the contrast enhancing agent to compare with subsequent data sets acquired from the same area of interest in the same patient subsequent to administration of the contrast enhancing agent, it is also possible to compare data sets acquired following administration of a contrast enhancing agent to empirically determined standard or control data sets.

Various data processing techniques may be advantageously used to assess the data collected in accordance with the present invention. Data may be analyzed and displayed in a variety of formats. Processing may include averaging or otherwise combining a plurality of data sets to produce control, subsequent or comparison data sets. Other optical data processing techniques include frequency domain methods such as Fourier or wavelet transformations of the optical data, or spatial domain methods such as convolutions, geometrical transformations, data differencing, and the like.

Data processing may also include amplification of certain signals or portions of a data set (e.g., areas of an image) to enhance the contrast seen in data set comparisons, and to thereby identify areas of abnormal tissue with a high degree of spatial resolution. For example, according to one embodiment, images are processed using a transformation in which data point brightness values are remapped to cover a broader dynamic range of values. A “low” value may be selected and mapped to zero, with all data point brightness values at or below the low value set to zero, and a “high” value may be selected and mapped to a selected value, with all data point brightness values at or above the high value mapped to the high value. Data having an intermediate brightness value, representing the dynamic changes in brightness indicative of changes in optical properties, may be mapped to linearly or logarithmically increasing brightness values. This type of processing manipulation is frequently referred to as a “histogram stretch” or point transformation, and can be used according to the present invention to enhance the contrast of data sets, such as images, representing differences in tissue type.

Data processing techniques may also be used to manipulate data sets to provide more accurate combined and comparison data. For example, patient movement, respiration, heartbeat or reflex activity may shift an area of interest during detection of optical properties and data collection. It is important that corresponding data points in data sets (such as corresponding pixels of an image) are precisely aligned, spatially, to provide accurate combined and comparison data. Such alignment may be accomplished manually by a practitioner having specialized skill and expertise, or using a variety of mechanical and/or mathematical means. Emr source(s) and optical detector(s) may, for example, be mounted in a relatively “fixed” condition in proximity to an area of interest. Optical markers may be fixed at an area of interest and detected as the data is collected to aid in manual alignment or mathematical manipulation. Motion artifacts may be reduced or substantially eliminated by timing the acquisition of data to the cycle of respiration, heartbeat, or the like, to normalize the data. Various processing techniques are described below and in the patents incorporated herein by reference.

Comparison data may be displayed in a variety of ways. For example, comparison data may be displayed in a graphical format that highlights optical differences differentiating normal from abnormal tissue. A preferred technique for presenting and displaying comparison data is in the form of visual images, or photographic frames, corresponding to the area of interest. This format provides a visualizable spatial location (two- or three-dimensional) of an area of interest that is useful for treatment, diagnosis and monitoring.

To enhance and provide better visualization of optical contrast between abnormal and normal tissue, comparison images may be processed to provide an enhanced contrast grey scale or even a color image. A look up table (“LUT”) may be provided, for example, that converts the gray scale values for each pixel to a different (higher contrast) gray scale value, or to a color value. Color values may map to a range of grey scale values, or color may be used to distinguish between positive-going and negative-going optical changes. In general, color-converted images provide higher contrast images that highlight changes in optical properties representing areas of malignant and normal tissue.

In operation, an area of interest in a patient is illuminated with electromagnetic radiation (emr) while one or a series of data points or data sets representing one or more optical properties of spatially definable areas in the area of interest is acquired. Data sets are acquired before and/or after the administration of a contrast enhancing agent. The area of interest may be exposed to the emr source(s), or may underlie skin, tissue, bone, dura, or the like, provided that the emr source(s) is selected and positioned to penetrate tissue overlying the area of interest. Alternatively, the area of interest may be located within tissue, and the emr source(s) and detector(s) selected and positioned for transillumination of the area of interest.

For operation in a static mode, a contrast enhancing agent is administered, such as by injection or topical application, to an area of interest, and a data set mapping one or more optical properties to spatial locations in the area of interest is acquired. Spatial locations evidencing contrasting optical properties highlight areas of normal and abnormal tissue. Application of a topical contrast enhancing agent such as iodine or a weak acidic or basic agent to the surface of an area of interest, such as cervical tissue, colo-rectal tissue, digestive system tissue, esophageal tissue, or the like, for example, is followed by illumination of the area of interest and detection of differential optical properties at different spatial locations within the area of interest. Similarly, injections of a contrast enhancing agent, such as indocyanine green, followed by illumination of the area of interest and detection of differential optical properties corresponding to different spatial locations within the area of interest, provides differentiation and spatial localization of abnormal tissue, such as cancerous tissue, from surrounding normal tissue.

Additionally, operation in a static mode may involve illumination and acquisition of data sets from two spatially separated locations and comparison of the data sets at one or more time points following administration of the contrast enhancing agent. Thus, for example, data representative of the optical properties of two different areas of breast tissue may be acquired at predetermined time intervals following administration of a contrast enhancing a gent, such as indocyanine green. One of the areas of interest is presumed to contain “normal” tissue. Data from the “normal” area of interest is compared to data from another area of interest to detect and spatially localize differential optical properties that are indicative of abnormal tissue.

Acquired data may be compared to control or background data during operation in a static or a dynamic mode. Control data may represent standards derived from optical properties of empirical data samples of desired tissue populations. Control data may thus be derived representing various normal tissue types as well as various abnormal tissue types, such as different types and grades of tumors. Comparison of data acquired following administration of a contrast enhancing agent to various types of control data may then provide identification and spatial localization of abnormal tissue, such as cancer, as well as typing of the abnormal tissue, such as identifying particular cancers, and grading of cancerous tissue. For abnormalities such as cancer, it may be desirable to compare multiple data sets acquired at intervals following administration of the contrast enhancing agent to control data to observe changes in the optical properties of tissue at the area of interest at predetermined time intervals following administration of the contrast enhancing agent. According to one embodiment, statistically significant, contrast enhanced changes in optical properties of tissue may be determined empirically for various types of tissue, cancers, contrast enhancing agents and the like. Comparison of a data set acquired following administration of a contrast enhancing agent to a control data set representing statistically significant changes provides identification of spatial locations within an area of interest evidencing statistically significant changes indicative of abnormalities.

In another dynamic mode, data acquired corresponding to an optical property of an area of interest prior to administration of a contrast enhancing agent represents control, or background, data. A series of data sets is preferably combined, for example by averaging, to obtain a control data set. The control data set is stored for comparison with data collected subsequently. Alternatively, control or background data corresponding to various conditions of tissue and areas of interest may be acquired, stored, and used for comparison. Control data sets may also be acquired, in real time, from an area of interest believed to contain normal tissue. A subsequent data set representing the corresponding optical property is acquired during a subsequent time period following administration of a contrast enhancing agent. A series of subsequent data sets is preferably combined, for example by averaging, to obtain a subsequent data set. Subsequent data sets are compared with one or more control data set(s) to obtain comparison data set(s), preferably difference data set(s). Comparison data sets are then examined for evidence of changes in optical properties representative of areas of abnormal versus normal tissue within an area of interest.

According to one embodiment, the methods and systems described herein may be employed to obtain three-dimensional information of an area of interest suspected to contain abnormal tissue by: (a) illuminating the area of interest with a least two different wavelengths of emr; (b) obtaining a sequence of control data sets corresponding to each wavelength of emr; (c) administering a contrasting enhancing agent; (d) obtaining a sequence of subsequent data sets for each wavelength of emr; (e) obtaining a series of comparison data sets for each wavelength of light by subtracting the control data set from the subsequent data set or alternatively, in the case of fluorescent dyes, subtracting the subsequent image from the control image; and (f) obtaining an enhanced comparison data set by ratioing the first comparison data set to the second comparison data set. Data corresponding to three dimensional spatial locations may also be acquired using multiple contrast enhancing agents having different spectral properties, and by employing optical tomography techniques. Specifically, photon time-of-flight techniques and frequency domain methods may also be used.

### BRIEF DESCRIPTION OF THE DRAWINGS

The methods and apparatus of the present invention will be described in greater detail below with reference to the following figures. The file of this patent contains at least one drawing executed in color. Copies of this patent will color drawing(s) will be provided by the Patent and Trademark Office upon request and payment of the necessary fee.

FIGS. 1A to 1C illustrates detection of optical changes indicative of neuronal activity in a human subject by direct cortical electrical stimulation, wherein FIG. 1A shows plots of the percent optical change for site 1 and site 3 regions; FIG. 1B show plots of the percent optical change per second in the spatial regions of site 1 and 4; and FIG. 1C shows plots of the percent optical change absorption per second in the spatial regions of sites 1 and 2.

FIG. 2 illustrates dynamic optical changes in tumor vs. non-tumor tissue through the intact skull.

FIG. 3 shows dynamic information of optical changes in tumor vs. non-tumor tissue.

FIG. 4 shows changes in optical properties due to dye uptake and clearance in tumor vs. non-tumor tissue.

FIG. 5 shows plots of percentage change of emr absorption per second in the spatial regions of sites 1, 2, and 3 of hind limb somatosensory cortex in an anesthetized rat and a plot of corresponding morphological measurements of the venule in the spatial region of site 2 FIGS. 6A and 6B shows time course and magnitude plots of dynamic optical changes in human cortex evoked in tongue and palate sensory areas and in Broca's area (language); wherein FIG. 6A shows the plots during the three tongue wiggling trials averaged spatially within sites. FIG. 6B shows the plots during one of the language naming trials averaged spatially within other sites.

FIGS. 7A and 7B illustrate a time course and magnitude of dynamic optical changes in human cortex evoked in Wernicke's area (language comprehension), wherein FIG. 7A shows plots of percentage change in optical absorption of tissue within certain sites and FIG. 7B shows percentage changes from other site.

FIG. 8 shows a schematic diagram illustrating an exemplary system of the present invention.

FIG. 9 shows a schematic diagram illustrating alternative exemplary systems of the present invention.

FIG. 10 shows a schematic diagram, illustrating the use of optical techniques of the present invention for identifying abnormal tissue.

### DETAILED DESCRIPTION OF THE INVENTION

Applicant's optical detection methods and systems are described in greater detail below with reference to certain preferred embodiments. The detailed descriptions of certain preferred embodiments are not intended to limit the scope of the applicant's invention as described herein and set forth in the appended claims. The following terms, as used in this specification and the appended claims, have the meanings indicated:

Area of Interest is an area of tissue that comprises the subject of acquired data sets. In a preferred embodiment, an area of interest is suspected of containing one or more sites of abnormal tissue. In some embodiments, an area of interest is believed to contain normal tissue and data acquired is used as control or background data.

Arithmetic Logic Unit (ALU) is a component that is capable of performing one or more processing (e.g., mathematical and logic) operation(s) (e.g., sum, difference, comparison, exclusive or multiply by a constant, etc.) on a data set.

Control Data is data representing one or more optical properties of an area of interest. Control data may be acquired during a “normal” or a predetermined period, such as prior to administration of a stimulus or a contrast enhancing agent. Control data may also be derived empirically or in real time from one or more “normal” tissue samples. The control data set establishes a “background” profile of optical properties for comparison with a data set acquired following administration of a stimulus or a contrast enhancing agent.

Charge Coupled Device (CCD) is a type of optical detector that utilizes a photosensitive silicon chip.

Comparison Data highlights spatial locations within an area of interest having different optical properties. The comparison may be of data points within a single data set, such as different spatial locations within an area of interest. Alternatively, a comparison may be made of data acquired subsequent to administration of a contrast enhancing agent, with control data, such as by adding, subtracting, or the like. The comparison data set is used to identify and/or locate areas of abnormalities indicated as areas of enhanced contrast.

Electromagnetic Radiation (emr) means energy having a wavelength of from about 450 to about 2500 nm. Emr illumination suitable for use with the optical detection techniques described herein is in the visible and infrared regions.

Frame is a digitized array of pixels.

Frame Buffer is a component that provides storage of a frame, such as a control image, a subsequent image or a comparison image.

Geometric Transformations can be used to modify spatial relationships between data points in a data set, such as pixels in an image. Geometric transformations are often called “rubber sheet transformations” because they can be viewed as the process of “printing” data, such as an image, on a sheet of rubber and stretching the sheet according to a predefined set of rules. As applied to optical detection, subsequent data sets can be viewed as having been distorted due to movement and it is desirable to “warp” these data sets so that they are spatially aligned with the control images. Geometric transformations are distinguished from “point transformations” in that point transformations modify a data point's (pixel's) value in a data set (an image) based solely upon that data point's (pixel's) value and/or location, and no other data point (pixel) values are involved in the transformation. Geometric transformations are described in Gonzalez and Wintz, Digital Image Processing, Addison-Wesley Publishing Co.: 1987.

Image is a frame or composition of frames representing one or more optical properties of an area of interest.

Optical Properties relate to various properties detectable in the useful range of emr (450-2500 nm) including, but not limited to, scattering (of various types), reflection, refraction, diffraction, absorption and extinction, birefringence, refractive index, Kerr effect and the like.

Optical Source is a device that illuminates an area of interest, permitting optical detection.

Optical Detector is a device capable of detecting one or more desired optical properties of an area of interest. Suitable optical detectors include any type of photon detector, such as photodiodes, photomultiplier tubes, cameras, video cameras, CCD cameras, and the like.

Optical Detection refers to the acquisition, and/or comparison, processing and display of data representative of one or more optical properties of an area of interest. Optical detection may involve acquisition, processing and display of data in the form of images, but need not.

Pixels are the individual units of an image in each frame of a digitized signal. The intensity of each pixel is proportional to the intensity of illumination before signal manipulation and corresponds to the amount of emr (photons) being scattered from a particular area of tissue corresponding to that particular pixel. An image pixel is the smallest unit of a digital image and its output intensity can be any value. A CCD pixel is the smallest detecting element on a CCD chip and its analog output is linearly proportional to the number of photons it detects.

Subsequent Data is data representing one or more optical properties of an area of interest during a monitoring period or subsequent to administration of a contrast enhancing agent.

Methods and systems of the present invention utilizing optical techniques may be used with or without a contrast enhancing agent to identify and spatially localize abnormal tissues and blood vessels, as w ell as blood characteristics, blood flow, blood volume, and blood vessel structure and function. According to one embodiment, optical detection techniques are used in conjunction with the administration of a contrast enhancing agent for diagnostic purposes to screen an area of interest to identify properties of tissues, blood flow and blood vessels and to locate abnormalities with a high degree of spatial resolution. Optical detection techniques may be used for examining an area of interest that is directly exposed to emr source(s) and detector(s), such as an area of interest exposed during a surgical procedure, or an area of interest exposed to an invasive or semi-invasive instrument, such as a laproscope, endoscope, probe, fiber optic cables, or the like. Alternatively, optical detection techniques of the present invention employ near infrared emr for non-invasively detecting cellular and tissue properties through and underneath intact skin, bone, tissue, and the like.

An area of interest located or embedded within tissue is examined by epi-illumination or transillumination following administration of a contrast enhancing agent. Techniques and systems of the present invention are used for identifying and spatially locating blood characteristics, blood flow and blood vessel abnormalities, including areas of ischemia, hypoxia, blood clotting, hematomas, increased or diminished blood flow resulting from disease, such as heart disease, cancer, neurological disorders, and the like. These systems are thus useful for diagnosis of many types of diseases and conditions characterized by blood characteristics, blood flow or blood vessel abnormalities, and are particularly useful as noninvasive or semi-invasive techniques for screening areas of interest to identify and spatially locate abnormalities. Additionally, methods and systems of the present invention may be used intraoperatively or interfaced with stereotaxic systems to assist medical personnel in spatially mapping blood flow during surgery and locating areas of increased or diminished blood flow, both during surgery, and during recovery.

Yet another application for methods and systems of the present invention involves in situ monitoring an area of interest to evaluate the progression, or recession, of a blood characteristics, blood flow or blood vessel abnormality in an area of interest, and to monitor, in situ, the effect of a treatment regimen or agent on an identified or suspected abnormality. Detection and monitoring of heart disease, blood flow blockages resulting from clots or damaged blood vessels, internal bleeding, hematomas, such as subdural hematomas, ischemia, hypoxia, and the like, are exemplary applications for methods and systems of the present invention. Methods and systems of the present invention may also be used to map blood flow in an area of interest.

Methods and systems of the present invention utilizing optical techniques and, optionally, involving the administration of a contrast enhancing agent to identify and localize tissue abnormalities characterized by different blood characteristics, blood flow, and blood vessel morphology, may be implemented for numerous applications. According to one embodiment, optical detection techniques used in conjunction with the administration of a contrast enhancing agent, are used for diagnostic purposes to screen an area of interest to identify whether abnormal tissue, specifically cancerous tissue characterized by different vascularization and blood flow, is present in the area of interest and, if so, to locate the cancerous tissue with a high degree of spatial resolution. Optical detection techniques may also be used to identify functional and dysfunctional areas in central and peripheral nervous tissue, since activated nervous tissue and dysfunctional nervous tissue are characterized by different blood characteristics or blood flow, optically detectable as differences in optical properties. Similar to the discussion above, these techniques may be used for examining an area of interest that is directly exposed to emr source(s) and detector(s), such as an area of interest exposed during a surgical procedure, or an area of interest exposed to an invasive or semi-invasive instrument, such as a laproscope, endoscope, probe, fiber optic cables, or the like. In this fashion, methods and systems of the present invention may be used for screening and diagnosis of various abnormalities, including cancers of the various organs and tissues, as well as various conditions manifesting central and peripheral nervous system tissue dysfunction. In particular, techniques and systems of the present invention are used for identifying and spatially locating blood characteristics, blood flow and blood vessel abnormalities, including areas of ischemia, hypoxia, blood clotting, hematomas, increased or diminished blood flow resulting from disease, such as heart disease, cancer, neurological disorders, and the like, as well as, cancers of the digestive system organs, including esophageal cancers, colorectal cancers, and the like; skin; reproductive organs, such as prostate, ovarian, uterine and cervical cancers, breast cancer; brain cancer; cancers of the lymphatic system and bone; and the like.

For some applications where the area of interest is directly exposed to emr source(s) and detector(s), permitting epi-illumination of the area of interest, topical application of a contrast enhancing agent may be preferred to other types of delivery systems. Thus, for example, topical application of a contrast enhancing agent such as iodine, or a weak acid or base such as weak acetic acid, to an area of interest such as cervical tissue, or to a surface of an internal organ or tissue, is followed by acquisition of one or more data sets indicative of one or more optical properties of the area of interest. Comparison of data points within the data set acquired following application of the contrast enhancing agent highlights areas of enhanced optical change indicative of changes in blood characteristics or blood flow and thereby highlights the location of abnormal tissue. Comparison of data set(s) acquired following administration of the contrast enhancing agent to control data indicative of one or more optical properties of normal tissue of the same type, or to control data acquired at the area of interest prior to application of the contrast enhancing agent, provides identification and spatial localization of abnormal tissue, particularly cancerous tissue, by highlighting the different optical properties of the tissue following administration of the contrast enhancing agent.

For other applications, the area of interest underlies skin, bone, tissue, dura, or the like and the emr source(s) provides longer wavelengths of emr that penetrate the overlying tissue to illuminate the area of interest. In general, emr in the near infrared range penetrates tissues sufficiently to provide illumination of areas of interest underlying skin, bone, dura and the like. According to one implementation of methods and systems of the present invention, an area of interest located or embedded within tissue may be examined by transilluminating the area of interest following administration of a contrast enhancing agent. This type of system is useful when a tissue surface overlying the area of interest can be illuminated with emr at a wavelength and at an intensity such that the emr travels through the area of interest and exits a tissue sample, and detectors can be arranged and positioned to detect the emr transmitted through the area of interest. This type of system is particularly useful for non-invasive detection or monitoring of cancerous tissue in breast tissue.

In one embodiment, a contrast enhancing agent is administered to provide perfusion of the area of interest. Initial detection of the contrast enhancing agent is manifest in many types of cancer tissue first, because cancer tissue is differently vascularized compared to non-cancerous tissue and many contrast enhancing agents therefore perfuse more rapidly into cancerous tissue than normal tissue. Solid tumor margins are generally the first morphological indications of cancer tissue detected by comparison of a control or background data set with a data set acquired from an area of interest containing cancerous tissue following administration of a contrast enhancing agent. In applications in which comparison data is output as an image and the detector is, for example, a camera, a comparison image shows darkened lines outlining a solid tumor mass.

Additionally, many contrast enhancing agents are cleared more slowly from cancerous tissue compared to non-cancerous, normal tissue. After the contrast enhancing agent has perfused throughout the area of interest in both normal tissue and tumor tissue, clearance of the contrast enhancing agent from tumor tissue is delayed compared to clearance of the contrast enhancing agent from normal, non-tumor tissue. This delay may be a consequence of the “leaking” nature of many blood vessels in cancerous tissue. This characteristic of the dynamics of perfusion of contrast enhancing agents in tumor compared to non-tumor tissue provides additional opportunities to identify and localize tumor tissue over the course of clearance of the contrast enhancing agent from the area of interest. Additionally, the more aggressive the tumor (higher tumor grade), the longer the contrast enhancing agent remains in the tumor tissue. It is therefore possible to grade malignant tissue using methods and systems of the present invention based on the rate of clearance of the contrast enhancing agent from the area of interest.

Methods and systems of the present invention may also be used to assist in the selection of tissue samples for biopsy. The selection of the biopsy sample is critical—every effort should be made to enhance the likelihood of including abnormal tissue. Yet, tissue biopsies are invasive and may affect important tissues, and therefore should be limited to reduce trauma and preserve function of the tissue. Lymph nodes are frequently biopsied, for example, in an effort to evaluate the extent and progression of various cancers. Administration of a contrast enhancing agent followed by illumination and optical detection to identify and spatially localize areas of abnormal tissue greatly aids in the selection of tissue samples to biopsy. Specifically, with the aid of an optical contrast enhancing agent and the optical techniques of the present invention, the likelihood of obtaining a biopsy sample including abnormal tissue is substantially increased. Optical source(s) and detector(s) may be incorporated in an invasive or non-invasive biopsy instrument, and the contrast enhancing agent may be administered in situ or in another fashion that provides application of the contrast enhancing agent in the area of interest.

Yet another application for methods and systems of the present invention involves in situ monitoring an area of interest to evaluate the progression, or recession, of a condition involving abnormal blood characteristics, blood flow, or blood vessels, such as pathological or tumor tissue, in an area of interest, and to monitor, in situ, the effect of a treatment regimen or agent on an identified or suspected area of interest, such as a tumor. Methods and systems of the present invention may be employed, for example, to provide frequent screening or monitoring of cancerous tissue to rapidly detect any progression that would benefit from additional or different treatment agents or regimen. Screening and monitoring may also be implemented to evaluate the need for additional testing using more expensive and less accessible techniques, such as MRI.

Diagnostic and monitoring procedures, optionally, involve administration of a contrast enhancing agent to an area of interest, followed by illumination and detection of one or more optical properties of the area of interest. A data set may be examined to identify areas of differential optical properties that may be indicative of normal or abnormal tissue. Comparison of data set(s) representing one or more optical properties of spatially defined locations in the area of interest following administration of the contrast enhancing agent may be made as described above. Such comparisons are preferably made continuously or at predetermined intervals following administration of the contrast enhancing agent to provide information relating to the time course of differential optical properties enhanced by the contrast enhancing agent at the area of interest.

The interaction between the emr and the contrast enhancing agent depends upon the specific agent being used. For example, in the case of a fluorescent dye, the preferred wavelength of emr is one which excites the dye, thereby causing fluorescence. However, for many contrast enhancing agents, such as indocyanine green, the preferred wavelength of emr is one which is absorbed by the dye.

The inventive methods and systems are superior to established tumor detection and localization techniques, such as MRI, because they are capable of distinguishing low grade tumors that generally are not distinguished using alternative techniques. Additionally, updated comparison data sets may be provided on a continuous or frequent basis during a surgical procedure, for example, by readministering a stimulus or a contrast enhancing agent. A stimulus or contrast enhancing agent may be administered on multiple occasions during a surgical procedure, for example, to examine an area of interest for functional or dysfunctional tissue, or for residual tumor tissue. For CNS tumors, MRI techniques can only image advanced stage tumors that have compromised the blood brain barrier. The present optical detection techniques, in contrast, are capable of detecting even low grade tumors that have not yet compromised the blood brain-barrier. Methods and systems of the present invention may be implemented using readily available equipment and provided at a substantially lower cost than traditional MRI and CT techniques. Methods and systems of the present invention are also preferable to existing X-ray techniques for screening for various cancers because they identify and locate cancerous tissue with substantially improved sensitivity and specificity.

The contrast enhancing agent may be any agent that provides differential contrast enhancement between normal and abnormal tissue. Emr-absorbing and fluorescent agents are suitable. Contrast enhancing agents having a short half-life are preferred for some applications, such as intraoperative applications. During surgical resection of a solid tumor, it is important that the agent be rapidly cleared from the area of interest so that additional doses of the contrast enhancing agent can be administered repeatedly to image residual tumor tissue. Agents suitable for use with the present invention include indocyanines, fluoresceins, hematoporphyrins, fluoresdamine and other dyes used for photodynamic treatment of tumor tissue, such as those available from Quadra Logic Technologies, Inc., iodine and weak acidic and basic agents (Vancouver, B.C.). Specific examples of agents which may be usefully employed with the present invention include indocyanine green, Photofrin®, NPe₆, BPD, Evans Blue, Biodipy® (available from Molecular Probes, Inc., Eugene, Oreg.) and combinations thereof. The delta 1,2 bicyclo [4,4,0] and delta.sup 1,6 bicyclo [4,4,0] functional dyes disclosed in U.S. Pat. Nos. 5,672,332 and 5,672,333 and similar agents may also be used with methods and systems of the present invention.

Yet another aspect of the inventive method and systems involves using an emr absorbing or fluorescent dye conjugated to a targeting molecule, such as an antibody, hormone, receptor, or the like. According to one embodiment, the targeting molecule is a monoclonal antibody or fragment thereof specific for surface marker of a tumor cell or a cell that circulates in the blood stream. When fluorescent agents are used, the area of interest is illuminated with emr containing excitation wavelengths of the fluorescent agent, but not emission wavelengths. This can be accomplished by use of a cutoff filter over the emr source. Preferably, the optical detector is coupled to an image intensifier or micro channel plate (e.g., KS-1381 Video Scope International, Wash DC) to increase the sensitivity of the system by several orders of magnitude and allow for visualization of cells having fluorescent dyes attached thereto. Examples of fluorescent dyes that can be conjugated to a targeting molecule include, for example, Cascade Blue, Texas Red and Lucifer. Yellow CH from Molecular Probes, Eugene, Oreg.

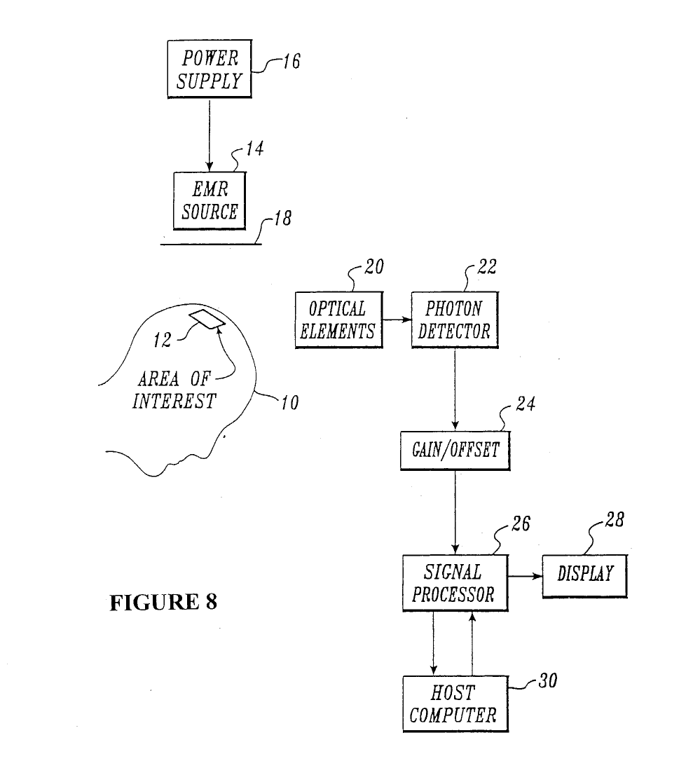

The inventive methods employ an apparatus comprising a source of emr, an optical detector for acquiring data representative of one or m ore optical properties of the area of interest, and data processing and display capability. The apparatus may be constructed as an integrated unit, or it may be used as a collection of components. The apparatus will be briefly described with reference to the schematic diagrams of FIGS. 8-10, and various components and features will then be described in greater detail.

FIG. 8 illustrates a human patient 10 whose neuronal tissue represents area of interest 12. As is described in greater detail below, area of interest 12 may be fully or partially exposed, or detection may be conducted through tissue such as bone and/or dura with proper selection of emr wavelengths. During optical imaging, area of interest 12 is illuminated by emr source 14 powered by regulated power supply 16. Emr is preferably directed through an optical filter 18 prior to contacting area of interest 12.

During optical detection, a light gathering optical element 20, such as a camera lens, endoscope, laparascope, optical fibers, or the like, and photon detector 22 are positioned to detect optical properties of area of interest 12. Signals representative of optical properties are processed, if desired, in a gain, offset component 24 and then conveyed to analog-to-digital (A/D) and digital signal processing hardware 26. Data representing optical properties, and particularly changes in optical properties, is displayed on display device 28. The optical detection, display and processing components are controlled by host computer 30.

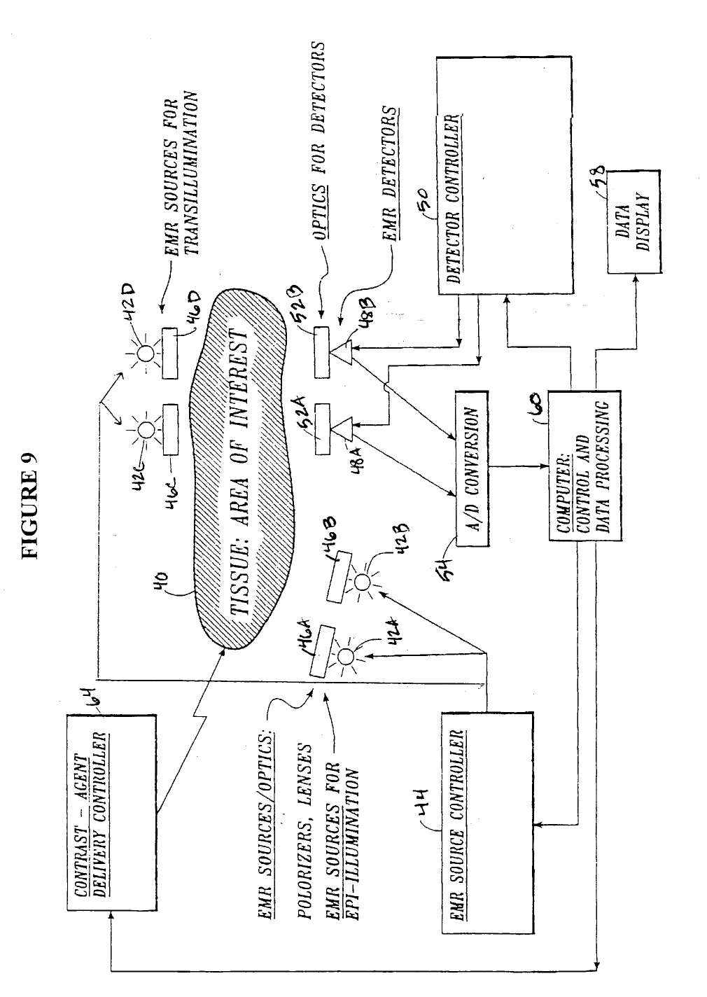

FIG. 9 shows another system of the present invention for operation in an epi-illumination or a transillumination mode. Tissue sample 40 represents the area of interest. An array of emr epi-illumination sources is represented by sources 42A and 42B controlled by emr source controller 44. Two emr epi-illumination sources are illustrated at 42A, 42B, but any number of emr sources may be used. An alternative array of emr transillumination sources 42C and 42D is controlled by emr source controller 44. Two emr transillumination sources are illustrated at 42C, 42D, but any number of emr sources may be used. Emr source controller 44 may provide controlled intensity, frequency modulation, wavelength modulation, and the like, and is itself controlled by central control and data processing unit 60. The emr illumination emitted by sources 42A-D may be intercepted by various optical elements 46A, 46B, 46C, 46D prior to impingement on the area of interest. Optical elements may include filters, diffusers, polarizers, lenses, and the like. Emr sources or associated optical elements may be spaced from a surface of the area of interest, as shown, or may directly contact the surface of an area of interest. In general, when an area of interest is exposed tissue, it is not necessary for the emr source or associated optical element to contact the area of interest. When the area of interest underlies tissue, such as bone or soft tissue, it may be desirable for the emr source or associated optical elements to contact an exterior surface in proximity to the underlying area of interest.

Emr detectors 48A and 48B are provided for detecting optical properties of spatially identifiable areas of the area of interest during illumination and following administration of a contrast enhancing agent. Two emr detectors are illustrated, but any number of emr detectors may be used. Emr detector controller 50 may provide various controls for data acquisition, including gain, offset, and various timing features, all of which are preferably controlled by central control and data processing unit 60. The emr detectors may be intercepted by various optical elements 52A, 52B prior to impingement on the area of interest. Optical elements may include polarizers, lenses, objectives, and the like. Emr detectors or associated optical elements may be spaced from a surface of the area of interest, as shown, or may directly contact the surface of an area of interest. Data acquired by emr detectors 48A, 48B is preferably converted from an analog to a digital form in A/D converter 54 before processing in central control and data processing unit 60.

Central control and data processing unit 60 may also control related events, such as the rate, timing and delivery of t he contrast enhancing agent. As shown schematically in FIG. 8, contrast enhancing agent delivery controller 64 is also controlled by central control and data processing unit 60. Various data processing and control features, which are described in detail herein, may be implemented by central control and data processing unit 60. Output data in a selected format is displayed on data display unit 58. Data may be displayed in the form of a graph or another format that highlights changes in the optical properties of spatial locations within the area of interest. According to a preferred embodiment, data display unit 62 displays a visual image of the area of interest, as described more fully and illustrated below.

Methods and systems of the present invention may be implemented to acquire data in a epi-illumination or transillumination mode, as shown, depending on the orientation of emr sources with respect to the emr detectors. For most applications, emr sources are located for epi-illumination or transillumination of the area of interest, but not both. For certain applications, however, it may be advantageous to provide both epi-illumination and transillumination of an area of interest.

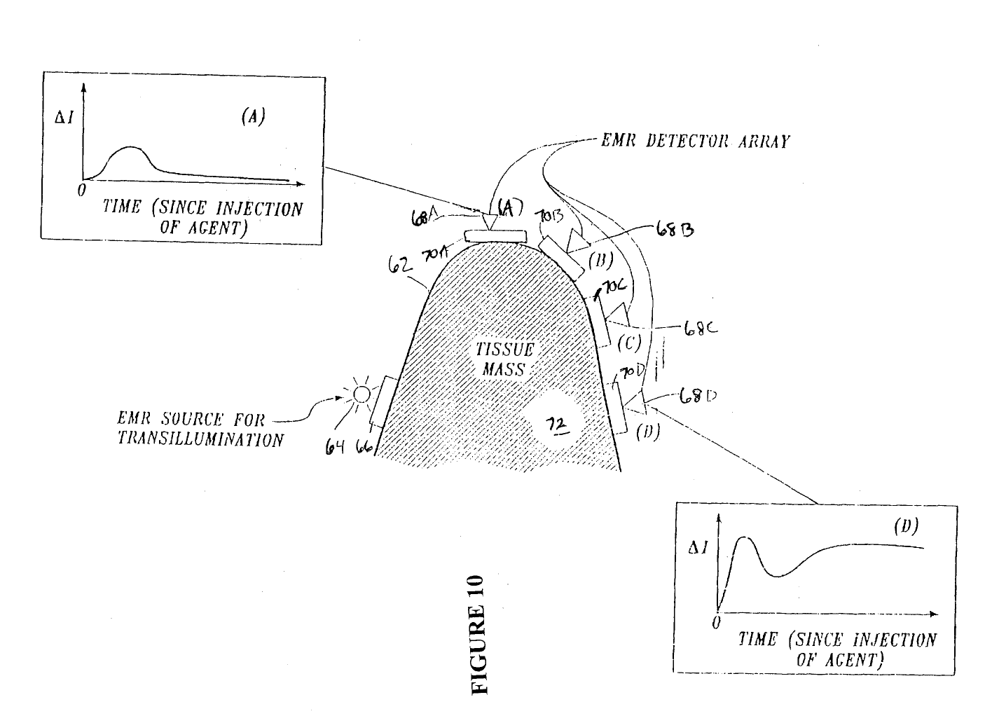

FIG. 10 shows, schematically, the acquisition of data using methods and systems of the present invention in a transillumination mode, as well as exemplary data output. Tissue mass 62 represents a breast tissue mass. Emr sources 64A-D and appropriate optical elements 66A-D are selected and positioned for transillumination of the tissue mass. An array of emr detectors 68A-D and appropriate optical elements 70A-D are selected and positioned to detect illumination transiting the tissue mass. Each of the emr detectors is positioned to acquire data from different areas of interest within the tissue mass. An appropriate number and arrangement of emr detectors is preferably provided so that the tissue mass can be screened in its entirety in a single operation. Emr sources and detectors and the appropriate optical elements are controlled and operated as described with reference to FIG. 9.

Following administration of a contrast enhancing agent, such as indocyanine green, data is acquired by each of the emr detectors at predetermined intervals. Plots of the change in intensity of light detected at areas of interest surveyed by emr detectors 68A and 68D over a time period following administration of the contrast enhancing agent are provided at insets A and D. The data acquired at emr detector 68A, shown in inset A, shows a gradual uptake of contrast enhancing agent as a gradual increase in the change in optical properties, followed by a gradual decrease in the change in optical properties, indicating the clearance of the contrast enhancing agent from the area of interest. The data acquired at emr detector 68D, shown at inset D, shows a rapid increase in the change in optical properties shortly after administration of the contrast enhancing agent, followed by a decrease in the change in optical properties, followed by a gradual and sustained increase in the change of optical properties in the area of interest. This data is illustrative of a contrast enhanced mass 72, such as a tumor, within tissue mass 62. The data from multiple emr detectors may alternatively be combined and output as a visual images that highlights and spatially localizes contrast enhanced masses, such as tumors. Methods and systems of the present invention using contrast enhancing agents may thus be implemented to provide identification and spatial localization of abnormal tissue, such as tumor tissue, in non-invasive manner.

Methods for imaging neuronal activity involve comparison of control data to data acquired during neuronal activity, inhibition or dysfunction. Neuronal tissue may be stimulated or inhibited without applying any external influence. Seizures, strokes, neuronal dysfunction and tissue non-viability are exemplary of such occurrences. Alternatively, intrinsic optical signals may be evoked by stimulating neuronal tissue using direct stimulation techniques or specific paradigms. Suitable paradigms are well known in the art and include, for example, presenting pictures of objects to a patient and asking the patient to name the object. Such naming exercises alter neuronal activity and produce an associated intrinsic signal.

An optical detector, such as a video CCD, is focused upon the area of interest during high intensity emr illumination. A first averaged image may be acquired, digitized and stored in a frame buffer. During an imaging study, it is important to update the averaged image frame frequently to account for patient movement and for tissue movements due to surgical manipulation. The area of interest is subsequently monitored at regular intervals, or an appropriate paradigm is administered. Subsequent image frames are acquired and stored, and subtractively compared to produce difference images (preferably, one or two per second) using the above-described processing means. The areas in which neuronal activity has occurred are indicated in the difference image. The difference image can be stored to allow the surgeon to study the area of interest in real time during an operation.

The present invention further provides a method for imaging of cortical functional areas and dysfunctional areas, such as those areas of severe epileptic activity. The method involves administering a paradigm to evoke an intrinsic signal for mapping a particular cortical function, or identifying an area of hyperactivity that is the location of epileptic activity in an epileptic patient. An epileptogenic area of the cortex is visualized as spontaneously more active and can be imaged by the inventive apparatus by mapping intrinsic signals of cortical activity. Retinal function and dysfunction may also be detected and monitored using the optical imaging techniques described herein.

The inventive apparatus and method may also be employed to image peripheral nerve damage and scarring. Nerves of the central and peripheral nervous system (PNS) are characterized by the ability to regenerate after damage. During operations to repair damaged peripheral or cranial nerves, one can image areas of nerve damage by imaging areas of blockage of intrinsic signals. For example, the nerve is exposed in the area of interest and then stimulated upstream of the site of damage. The active nerve pathway is imaged by intrinsic signals in the processed difference image after stimulation. The site of nerve damage or blockage is evidenced by an abrupt end or diminution to the intrinsic signal. In this way, the surgeon is able to obtain real time information on the precise location of nerve damage and to correct the damage, if possible.

High resolution detection of dynamic optical properties indicative of physiological activity may be accomplished without using dyes or other types of contrast enhancing agents according to the methods and apparatus of the present invention, as evidenced by the examples described herein. Many of the assessment techniques and apparatus of the present invention are physiologically noninvasive, in that detection and analysis of geometrical and/or intrinsic optical information does not require direct contact of the area of interest with any agents such as dyes, oils, devices, or the like. For particular applications, it may, however, be useful to administer one or more contrast enhancing agents that amplify differences in an optical property being detected as a function of the physiological state prior to acquiring subsequent data and generating a comparison. The use of contrast enhancing agents is described in detail, with reference to optical imaging of tumor and non-tumor tissue, in U.S. Pat. No. 5,465,718 and U.S. Pat. No. 5,438,989, which are incorporated by reference herein in their entireties.

Normally, areas of increased neuronal activity exhibit an increase of the emr absorption capacity of neuronal tissue (i.e., the tissue gets darker if visible lights is used for emr illumination, or an intrinsic signal increases in a positive direction). Similarly, a decrease in neuronal activity indicates a decrease of emr absorption capacity of the tissue (i.e., the tissue appears brighter, or intrinsic signals become negative). For example, image A is a subsequent averaged image and image B is an averaged control image. Normally, when a pixel in image A is subtracted from a pixel in image B and a negative value results, this value is treated as zero. Hence, difference images cannot account for areas of inhibition. The present invention provides a method for identifying both negative and positive intrinsic signals, by: (a) subtracting image A (a subsequent averaged image) from image B (an averaged control image) to create a first difference image, whereby all negative pixel values are zero; and (b) subtracting image B from image A to create a second difference image whereby all negative pixel values are zero; and adding the first and second difference images to create a “sum difference image.” The sum difference image shows areas of increased activity and show areas of less activity or inhibition. Alternatively, one can overlay the first difference image on the second difference image. Either method provides an image of increased neuronal activity and decreased neuronal activity. The difference output may be superimposed upon the real time analog video image to provide a video image of the area of interest (e.g., cortical surface) superimposed with either a gray-scale or a color-coded difference frame, in frozen time, to indicate where there are intrinsic signals in response to some stimulus or paradigm.

Diagnostic and monitoring procedures involve administration of a stimulus, followed by illumination and detection of one or more optical properties of spatially resolved areas of the area of interest. A data set may be examined to identify areas of differential optical properties that may be indicative of active or dysfunctional tissue. Comparison of data set(s) representing one or more optical properties of spatially defined locations in the area of interest following administration of a stimulus or another event producing central or peripheral nervous system activity may be made as described above. Such comparisons are preferably made continuously or at predetermined intervals following administration of stimulus to provide information relating to the time course of differential optical properties at the area of interest. Updated comparison data sets may be provided on a continuous or frequent basis during a surgical procedure, for example, by readministering the stimulus. The stimulus may be administered on multiple occasions during a surgical procedure, for example, to spatially locate areas of dysfunction or areas demonstrating various functional activities. Methods and systems of the present invention may be implemented using readily available equipment and provided at a substantially lower cost than traditional MRI and CT techniques.

A contrast enhancing agent that provides differential contrast enhancement between active and dysfunctional tissue of the central and peripheral nervous systems may, optionally, be employed. Emr-absorbing and fluorescent agents are suitable. Contrast enhancing agents having a short half-life are preferred for some applications, such as intraoperative applications. Agents suitable for use with the present invention include indocyanines, fluoresceins, hematoporphyrins, fluoresdamine and other dyes used for photodynamic treatment of tumor tissue, such as those available from Quadra Logic Technologies, Inc. (Vancouver, B.C.). Specific examples of agents which may be usefully employed with the present invention include indocyanine green, Photofrin®, NPe₆, BPD, Evans Blue, Biodipy® (available from Molecular Probes, Inc., Eugene, Oreg.) and combinations thereof. The delta 1,2 bicyclo [4,4,0] and delta.sup. 1,6 bicyclo [4,4,0] functional dyes disclosed in U.S. Pat. Nos. 5,672,332 and 5,672,333 and similar agents may also be used with methods and systems of the present invention.

Yet another aspect of the inventive method and systems involves using an emr absorbing or fluorescent dye conjugated to a targeting molecule, such as an antibody, hormone, receptor, or the like. According to one embodiment, the targeting molecule is a monoclonal antibody or fragment thereof specific for a surface marker of a central or peripheral nervous system cell. When fluorescent agents are used, the area of interest is illuminated with emr containing excitation wavelengths of the fluorescent agent, but not emission wavelengths. This can be accomplished by use of a cutoff filter over the emr source. Preferably, the optical detector is coupled to an image intensifier or micro channel plate (e.g., KS-1381 Video Scope International, Wash DC) to increase the sensitivity of the system by several orders of magnitude and allow for visualization of cells having fluorescent dyes attached thereto. Examples of fluorescent dyes that can be conjugated to a targeting molecule include, for example, Cascade Blue, Texas Red and Lucifer Yellow CH from Molecular Probes, Eugene, Oreg.

The inventive methods employ an apparatus comprising a source of emr, an optical detector for acquiring data representative of one or more optical properties of the area of interest, and data processing and display capability. The apparatus may be constructed as an integrated unit, or it may be used as a collection of components. The apparatus depicted in FIGS. 8-10, and described above, are equally applicable to the present embodiment, i.e., detecting abnormalities in central and peripheral nervous systems.

In addition, acquisition of data using methods and systems of the present invention may be employed in a transillumination mode. A tissue mass may represent an appendage, such as an arm, wrist, finger, or the like. One or more emr sources and appropriate optical elements are selected and positioned for transillumination of the tissue mass. An array of emr detectors and appropriate optical elements are selected and positioned opposite to one or more emr sources and appropriate optical elements to detect illumination transiting the tissue mass. Each of the emr detectors is positioned to acquire data from different areas of interest within the tissue mass. An appropriate number and arrangement of emr detectors is preferably provided so that the tissue mass can be screened in its entirety in a single operation. Emr sources and detectors and the appropriate optical elements are controlled and operated as described with reference to FIG. 9.

Following stimulation of a nerve underlying the emr source/detector array, data is acquired by each of the emr detectors at predetermined intervals. Plots of the change in intensity of light detected at areas of interest surveyed by emr detectors 68A and 68D over a time period following administration of the contrast enhancing agent are provided at insets A and D. The data acquired at emr detector 68A, shown in inset A, shows no change in the intensity of emr detected. The data acquired at emr detector 68D, shown at inset D, shows a spike in the change in intensity that corresponds to nerve stimulation, followed by a decrease in the changes in optical properties, followed by no change. This data is illustrative of a nerve 72, within tissue mass 62 having normal nerve function in proximity to detector 68D, and no nerve function in proximity to detector 68A. The data from multiple emr detectors may alternatively be combined and output as a visual images that highlight and spatially, localize nerve activity and dysfunction. Methods and systems of the present invention may thus be implemented to provide identification and spatial localization of abnormal nerve tissue in non-invasive manner.

One or more emr source(s) is used for illuminating the area of interest during acquisition of data representing one or more optical properties. The emr source(s);may provide epi-illumination or transillumination, as described above, depending on the relationship between the emr source(s) and detector(s). The emr source(s) may-illuminate an area of interest directly, as when tissue is exposed during or in connection with surgery, or it may be utilized to illuminate an area of interest indirectly through adjacent or overlying tissue such as bone, dura, skin, tissue, muscle and the like. Emr sources employed in methods and systems of the present invention preferably provide high intensity illumination. Exemplary emr sources include tungsten-halogen lamps, lasers, light emitting diodes, filtered incandescent sources, and the like. Cutoff filters that selectively pass all wavelengths above or below a selected wavelength may be employed. According to one embodiment, a preferred cutoff filter excludes all wavelengths below about 695 nm. An alternative to using cutoff filters involves administration of a first contrast enhancing agent prior to administration of a second, different contrast enhancing agent that acts as a tissue filter of emr to provide a filter in the area of interest. In this instance, it is desirable to utilize a contrast enhancing agent that remains with tumor or normal tissue for a prolonged period of time. According to another embodiment, illumination is provided through fiber optic strands using a beam splitter controlled by a D.C. regulated power supply (Lambda, Inc.). The emr source(s) may be operated in a continuous illumination mode, or in frequency modulated modes.

Preferred emr wavelengths for use with methods and systems of the present invention include wavelengths of from about 450 nm to about 2500 nm and, most preferably, wavelengths of the near infrared spectrum of from about 700 nm to about 2500 nm. Generally, longer wavelengths (e.g., approximately 800 nm) are employed to analyze deeper areas of tissue. Moreover, if a comparison is made between a data set obtained at 500 nm emr and a data set obtained at 700 nm emr, the difference comparison will show an optical slice of tissue. Selected wavelengths of emr may also be used, for example, when various types of contrast enhancing agents are administered.

According to one embodiment, the area of interest is uniformly illuminated to permit adjustment of the signal over a full dynamic range, as described below. Non-uniformity of illumination is generally caused by fluctuations of the illumination source and intensity variations resulting from the three-dimensional nature of the surface of the area of interest. More uniform illumination can be provided over the area of interest, for example, using diffuse lighting, mounting a wavelength cutoff filter in front of the optical detector and/or emr source, or combinations thereof. Fluctuation of the illumination source itself is preferably addressed by using a light feedback mechanism to regulate the power supply of the illumination source. Additionally, optically transparent plate may contact and cover the area of interest to provide a flatter, more uniform contour. The use of a plate or another mechanical aid to stabilize tissue in an area of interest also diminishes tissue movement during data acquisition. Fluctuations in illumination can be compensated for by using image processing algorithms, including placing a constant shade grey image marker point at the area of interest as a control point.

Methods and systems of the present invention may also usefully employ non-continuous illumination and detection techniques. For example, short pulse (time domain), pulsed time, and amplitude modulated (frequency domain) illumination sources may be used in conjunction with suitable detectors (See Yodh A and Chance B, Physics Today, March, 1995). Frequency domain illumination sources typically comprise an array of multiple source elements, such as laser diodes, with each element modulated at 180° out of phase with respect to adjacent elements (See, Chance B et al., Proc. Natl. Acad. Sci. USA 90, 3423-3427, 1993). Two-dimensional arrays, comprising four or more elements in two orthogonal planes, can be employed to obtain two-dimensional localization information. Such techniques are described in U.S. Pat. Nos. 4,972,331 and 5,187,672 which are hereby incorporated herein by reference in their entireties.

Time-of-flight and absorbance techniques (Benaron, D A and Stevenson D K, Science 259:1463-1466, 1993) may also be usefully employed in the present invention. In yet another embodiment of the present invention, a scanning laser beam may be used in conjunction with a suitable detector, such as a photomultiplier tube, to obtain high resolution images of an area of interest.

Illumination with a part of the infrared spectrum allows for imaging intrinsic signals through tissue overlying or adjacent the area of interest, such as dura, skull, skin, soft tissue, or the like. One exemplary infrared emr source suitable for imaging through tissue overlying or adjacent the area of interest is a Tunable IR Diode Laser from Laser Photonics, Orlando, Fla. When using this range of far infrared wavelengths, the optical detector is preferably provided as an infrared (IR) detector. IR detectors are made from materials such as indium arsenide, germanium and mercury cadmium telluride, and are generally cryogenically cooled to enhance their sensitivity to small changes in infrared radiation. One example of an IR imaging system which may be usefully employed in the present invention is an IRC-64 infrared camera (Cincinnati Electronics, Mason, Ohio).

One or more optical detector(s) is provided for acquiring a signal representative of one or more optical properties at spatially resolved areas within the of the area of interest. Any photon detector may be employed as an optical detector. Suitable detectors include photodiodes, photomultiplier tubes, photon intensifiers, cameras, video cameras, photon sensitive semiconductor devices, CCD cameras, and the like. Specialized detectors suitable for detecting selected optical properties and having high sensitivity may be employed. One preferred optical detector for acquiring data in the format of an analog video signal is a charge coupled device (CCD) video camera. One suitable device is a CCD-72 Solid State Camera (Dage-MTI Inc., Mich. City, Ind.). Another suitable device is a COHU 6510 CCD Monochrome Camera with a COHU 6500 electronic control b ox (COHU Electronics, S an Diego, Calif.). In some cameras, the analog signal is digitized 8-bits deep on an ADI board (analog-to-digital board). The CCD may be cooled, if necessary, to reduce thermal noise.

Data processing is an important feature of the optical detection techniques and apparatus of the present invention. Optical data processing techniques include frequency domain methods such as Fourier or wavelet transformations of the optical data, spatial domain methods such as convolutions, geometrical transformations, data differencing, and the like.

In use, for example, a CCD apparatus is preferably adjusted (at the level of the analog signal and before digitizing) to amplify the signal and spread the signal across the full possible dynamic range, thereby maximizing the sensitivity of the apparatus. Specific methods for detecting optical signals with sensitivity across a full dynamic range are described in detail in the patents incorporated herein by reference. Means for performing a histogram stretch of the difference frames (e.g., Histogram/Feature Extractor HF 151-1-V module, Imaging Technology, Woburn Mass.) may be provided, for example, to enhance each difference image across its dynamic range. Exemplary linear histogram stretches are described in Green, Digital Image Processing: a systems approach, Van Nostrand Reinhold: New York, 1983. A histogram stretch takes the brightest pixel, or the pixel with the highest value in the comparison image, and assigns it the maximum value. The lowest pixel value is assigned the minimum value, and every other value in between is assigned a linear value (for a linear histogram stretch) or a logarithmic value (for a log histogram stretch) between the maximum and minimum values. This allows the comparison image to take advantage of the full dynamic range and provide a high contrast image that clearly identifies areas of tumor tissue.

Noise (such as 60 Hz noise from A.C. power lines) is filtered out in the control unit by an analog filter. Additional adjustments may further enhance, amplify and condition the analog signal from a CCD detector. One means for adjusting the input analog signal is to digitize this signal at video speed (30 Hz), and view the area of interest as a digitized, image that is subsequently converted back to analog format.

It is important that data, such as consecutive data sets acquired from of a particular area of interest, be aligned so that data corresponding to the same spatial location is compared. If an averaged control data set and a subsequent data set are misaligned prior to comparison, artifacts will be present and the resulting comparison data set will amplify noise and edge information. Data set misalignment can be caused by patient motion, heartbeat, respiration, and the like. Large patient movements may require realignment of the optical detector and acquisition of a new control data set. It is possible, however, to compensate for small patient or tissue movements using various controls, mechanical or computational means, or a combination of all of these means. The optical detector and emr source may be provided as an integral unit, for example, to reduce relative motion and improve the integrity of data sets. Other techniques for maintaining the optical detector and the illumination source in a constant orientation with respect to the area of interest may also be employed.

Real-time motion compensation and geometric transformations may be used to align corresponding data. Simple mechanical translation of data or more complex (and generally more accurate) geometric transformation techniques can be implemented, depending upon the input data collection rate and amount and type of data processing. For many types of data sets, it is possible to compensate geometrically by translating the image by the x-y plane. In order for an algorithm such as this to be feasible, it must be computationally efficient (preferably implementable in integer arithmetic), memory efficient, and robust with respect to changes in ambient light.

Functional control points can be placed in the area of interest and triangulation-type algorithms used to compensate for movements of these control points. Control points can be placed directly in the area of interest. Goshtasby (“Piecewise Linear Mapping Functions for Image Registration,” Pattern Recognition 19:459-66, 1986) describes a method whereby an image is divided into triangular regions using control points. A separate geometrical transformation is applied to each triangular region to spatially register each control point to a corresponding triangular region in a control data set.

“Data warping” techniques may be employed whereby each subsequent data set is registered geometrically to the control data set to compensate for movement. Data warping techniques described, for example, in Wolberg, Digital Image Warping, IEEE Computer Society Press: Los Alimitos, Calif., 1990, may be used. Data warping techniques may further indicate when movement has become too great for effective compensation and a new control data set must be acquired.

Motion artifacts such as patient respiration, heartbeat or reflex activity may also be reduced or substantially eliminated by timing the acquisition of data to the cycle of respiration, heartbeat, or the like, to normalize the data. Acquisition of data may also be controlled to provide data acquisition at predetermined time points following administration(s) of the contrast enhancing agent.

The data processing function is generally operated and controlled by a host computer. The host computer may comprise any general computer (such as an IBM PC type with an Intel 386, 486 Pentium or similar microprocessor or Sun SPARC) that is interfaced with the emr source and/or optical detector and directs data flow, computations, data acquisition and output, and the like. Thus, the host computer controls acquisition and processing of data and provides a user interface.

According to a preferred embodiment, the host computer comprises a single-board embedded computer with a VME64 interface, or a standard (IEEE 1014-1987) VME interface, depending upon bus band width considerations. Host computer boards which may be employed in the present invention include, for example, Force SPARC/CPU-2E and HP9000 Model 7471. The user interface can be, for example, a Unix/X-Window environment. The processing board can be, for example, based upon Texas Instruments' MVP and other chips providing real-time image averaging, registration and other processing necessary to produce high quality comparison data. According to a preferred embodiment, comparison data is output in an image format. T he processing board may also drive, for example, a 120×1024 RGB display to show a sequence of difference images over time with pseudo-color mapping to highlight tumor tissue. Preferably, a second monitor is used for the host computer to increase the overall screen real estate and smooth the user interface. The processing board (fully programmable) can support a VME64 master interface to control data transactions with the other boards. Lastly, a peripheral control board can provide electrical interfaces to control mechanical interfaces from the host computer. Such mechanical interfaces can include, for example, the light source and optical detector control box.

A real-time data acquisition and display system, for example, may comprise four boards for acquisition, image processing, peripheral control and host computer. A minimal configuration with reduced processing capabilities may comprise just the acquisition and host computer boards. The acquisition board comprises circuitry to perform real-time averaging of incoming video frames and allow readout of averaged frames at a maximum rate bus. A VME bus is preferred because of its high peak bandwidth and compatibility with a multitude of existing VME products. The acquisition board should also support many different types of optical detectors via a variable scan interface. A daughter board may support the interfacing needs of many different types of optical detectors and supply variable scan signals to the acquisition motherboard. Preferably, the unit comprises a daughter board interfacing to an RS-170A video signal to support a wide base of cameras. Other camera types, such as slow scan cameras with a higher spatial/contrast resolution and/or better signal to noise ratio, can be developed and incorporated in the inventive device, as well as improved daughter boards to accommodate such improved cameras.

According to a preferred embodiment, data, such as analog video signals, are continuously processed using, for example, an image analyzer (e.g., Series 151 Image Processor, Imaging Technology, Inc., Woburn Mass.). An image analyzer can receive and digitize an analog video signal with an analog to digital interface and perform such a function at a frame speed of about 1/30th of a second (e.g., 30 Hz or “video speed”). Processing the signal involves first digitizing the signal into a series of pixels or small squares assigned a value (in a binary system) dependent upon the number of photons (i.e., quantity of emr) being detected from the part of the area of interest assigned to that pixel. For example, in a standard 512×512 image from a CCD camera, there would be 262,144 pixels per image. In an 8 bit system, each pixel is represented by 8 bits corresponding to one of 256 levels of grey.

The signal processing unit preferably includes a programmable look-up table (e.g., CM150-LUT16, Imaging Technology, Inc., Woburn, Mass.) initialized with values for converting grey coded pixel values, representative of a black and white image, to color coded values based upon the intensity of each grey coded value. This can provide image enhancement via an image stretch. An image stretch is a technique whereby the highest and lowest pixel intensity values used to represent each of the pixels in a digital image frame are determined over a region of the image frame which is to be stretched. Stretching a selected region over a larger range of values permits, for example, easier identification and removal of relatively high, spurious values due to noise (e.g., glare).

The processing unit may further include a plurality of frame buffers having frame storage areas for storing frames of digitized data received from the analog/digital interface. The frame storage area comprises at least one megabyte of memory space, and preferably at least 8 megabytes of storage space. An additional 16-bit frame storage area is preferred as an accumulator for storing processed image frames having pixel intensities represented by more than 8 bits. The processing unit preferably includes at least three frame buffers, one for storing the control data set, another for storing the subsequent data set, and a third for storing a comparison data set.

According to preferred embodiments, the processing unit further comprises an arithmetic logic unit (e.g., ALU-150 Pipeline Processor) for performing arithmetical and logical functions on data located in one or more frame buffers. An ALU may, for example, provide data averaging in real time. Newly acquired digitized image may be sent directly to the ALU and combined with control data stored in a frame buffer. A 16 bit result can be processed through an ALU, which will divide this result by a constant (i.e., the total number of data sets). The output from the ALU may be stored in a frame buffer, further processed, or used as an input and combined with another image.

The comparison (e.g., difference) data is, preferably, further processed to smooth out the output comparison and remove high frequency noise. For example, a low pass spatial filter can block high spatial frequencies and/or low spatial frequencies to remove high frequency noise at either end of the dynamic range. This provides a smoothed-out processed difference data set in digital format. The digitally processed difference data set in the form of an image can, for example, be color-coded by assigning a spectrum of colors to differing shades of grey. This image is then converted back to an analog image (by an ADI board) and displayed for a real time visualization of differences between the control data set(s) and subsequent data set(s). Moreover, the processed difference data set can be superimposed over the analog data set to display specific tissue sites where a contrast enhancing agent may have a faster uptake.

Processing speed may be enhanced by adding a real time modular processor or faster CPU chip to the image processor. One example of a real time modular processor which may be employed in the present invention is a 150 RTMP-150 Real Time Modular Processor (Imaging Technology, Woburn, Mass.).

The processing unit may further include an optical disk for storing digital data, a printer for providing a hard copy of the digital and/or analog data and a display, such as a video monitor to permit the physician to continuously monitor the comparison data output.

A single chassis may house all of the modules necessary to provide optical detection of tissue abnormalities according to the present invention. The necessary components, whether or to whatever degree integrated, may be installed on a rack that is easily transportable within and between operating and hospital rooms along with display monitors and peripheral input and output devices.

According to another embodiment, optical screening and monitoring devices of the present invention are provided in a modular design integrating a centralized data acquisition, processing and display device with interchangeable optical sources and detectors suitable for use in screening particular areas of interest. Using a modular design, the centralized data acquisition, processing and display device may be used in connection with one set of optical source(s) and detector(s) to assist in tissue sampling, or acquiring a biopsy for diagnostic evaluation in conjunction with one or more optical source(s) and detector(s) adapted for use with or mounted on a biopsy probe or another biopsy source(s) and instrument. Cervical cancer screening or monitoring may be provided using the centralized data acquisition, processing and display device with another set of optical source(s) and detector(s) mountable, for example, on a standard probe or other instrument used in gynecological examinations. Similarly, another set of optical source(s) and detector(s) may be mounted on a laparascope or endoscope and interfaced with the centralized data acquisition, processing and display device to provide screening for abnormal tissues in internal organs and tissues. Yet another set of optical source(s) and detector(s) may be provided for transilluminating an area of interest, such as breast tissue, and interfaced with the centralized data acquisition, processing and display device to detect abnormalities within tissue. Multiple emr sources and detectors for use in a transillumination mode may be interfaced and provided in a flexible arrangement that conforms to the surface contours of the area of interest. Alternatively or additionally, one or more emr sources and detectors may be implanted in an area of interest and interfaced with a centralized data acquisition, processing and display device continuously or at intervals to monitor the area of interest. The following examples are provided for illustration of specific embodiments and are not intended to limit the methods and systems of the present invention, as described and claimed herein.

### EXAMPLE 1

This example illustrates optical changes indicative of neuronal activity in a human subject by direct cortical electrical stimulation. A human cortex just anterior to face-motor cortex was tested with one recording (R) and two stimulating (S) electrodes, and four sites, site 1, site 2, site 3 and site 4, where average percent changes in corresponding optical properties were determined as described in Example 1.

Surface electrical recordings (surface EEG, ECOG) were correlated with optical changes. Intrinsic optical changes were evoked in an awake patient during stimulating-electrode “calibration”. Four stimulation trials were sequentially applied to the cortical surface, each stimulation evoking an epileptiform after-discharge episode. A stimulation trial consisted of: (1) monitoring resting cortical activity by observing the output of the recording electrodes for a brief period of time; (2) applying an electric current via the stimulation-electrodes to the cortical surface at a particular current for several seconds; and (3) monitoring the output of the recording electrodes for a period of time after stimulation has ceased.

The cortex was evenly illuminated by a fiber optic emr passing through a beam splitter, controlled by a D.C. regulated power supply (Lambda, Inc.) and passed through a 695 nm long pass filter. Images were acquired with a CCD camera (COHU 6500) fitted to the operating microscope with a specially modified cineadaptor. The cortex was stabilized with a glass footplate. Images were acquired at 30 Hz and digitized at 8 bits (512×480 pixels, using an Imaging Technology Inc. Series 151 system, Woburn, Mass.). Geometric transformations were applied to images to compensate for small amounts of patient motion (Wohlberg, Digital Imaging Warping, IEEE Computer Society: Los Alamitos, Calif., 1988). Subtraction of images collected during the stimulated state (e.g., during cortical surface stimulation, tongue movement or naming) from those collected during a control state with subsequent division by a control image resulted in percentage difference maps. Raw data (i.e., no digital enhancement) were used for determining the average optical change in specified regions (average sized sites was 30×30 pixels or 150-250 μm²). For pseudocolor images, a linear low pass filter removed high frequency noise and linear histogram transformations were applied. Noise was defined as the standard deviation of fluctuations in sequentially acquired control images as 0.003-0.009.

A series of images (each image consisting of an average of 128 frames acquired at 30 Hz) was acquired during each of the four stimulation trials. A current of 6 mA was used for the first three stimulation trials, and 8 mA for the fourth. After a sequence of 3-6 averaged control images were acquired, a bipolar cortical stimulation current was applied (either 6 mA or 8 mA) until epileptiform after discharge activity was evoked (as recorded by the surface electrode). Images were continuously acquired throughout each of the four stimulation trials.

The percentage change in absorption of light for each pixel was calculated for each image acquired during the four stimulation trials. The average percentage changes over the four areas were plotted graphically for comparison and analysis of the dynamic changes occurring in these four spatial areas as percentage optical change per second.

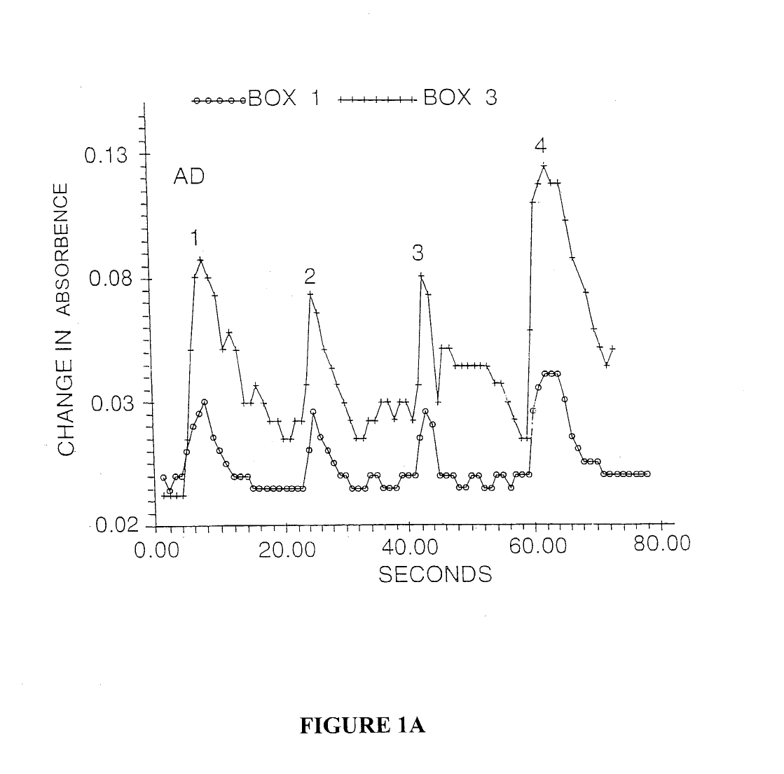

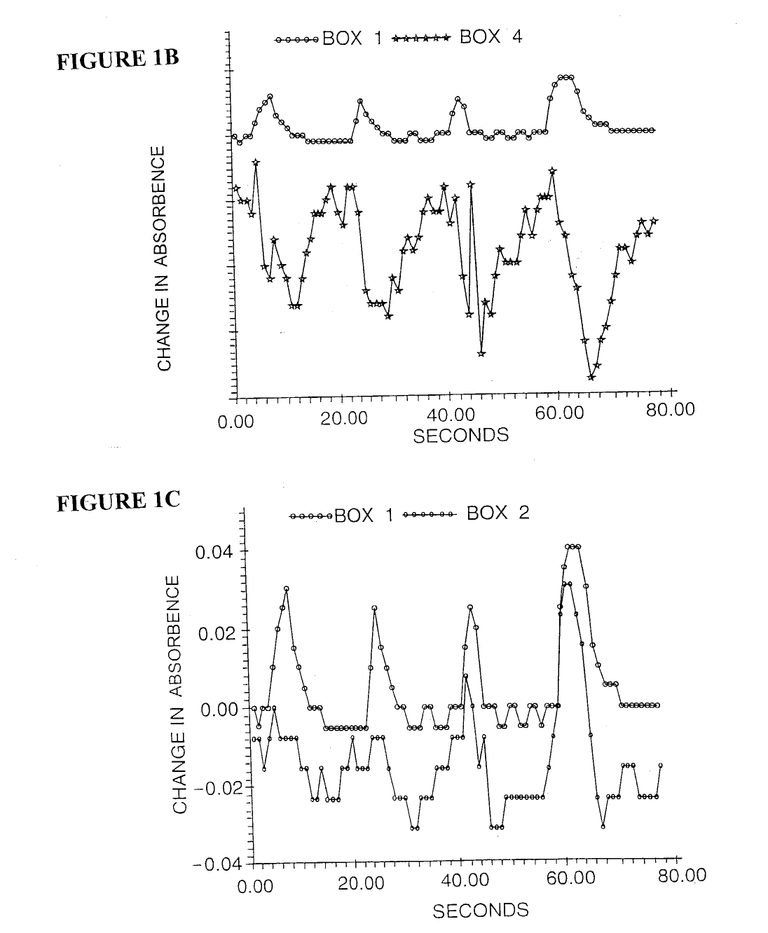

Site 1 overlaid and area of cortical tissue between the two stimulating electrodes. Site 3 was overlying the-area-of the epileptic focus. FIG. 1A shows plots of the percent optical change for site 1 and site 3 regions. The peak change was during the fourth stimulation trial (at 8 mA), in which the greatest amount of stimulating current had induced the most prolonged epileptiform after-discharge activity. The changes within site 3 were greater and more prolonged than those of site 1.

Site 4 overlaid a blood vessel. FIG. 1B show plots of the percent optical change per second in the spatial regions of site 1 and 4. The optical changes within site 4 were much larger and in the opposite direction of site 1. Also, these changes were graded with the magnitude of stimulating current and after-discharge activity. The changes in site 4 were most likely due to changes of the blood-flow rate within a blood vessel. This data demonstrates that the methods and apparatus of the present invention can be used to simultaneously monitor cortical activity and blood-flow.

FIG. 1C shows plots of the percent optical change absorption per second in the spatial regions of sites 1 and 2. The areas of site 1 and site 2 were nearby each other, but their optical changes was in the opposite direction during the first three stimulation trials using 6 mA current. Negative going changes found within the region of site 2 indicated that the methods and apparatus of the present invention may be used to monitor inhibition of cortical activity as well as excitation.

The optical changes between the stimulating electrodes at site 1 and near the recording electrode at site 3 showed a graded response to the intensity and duration of each after-discharge episode. The spatial extent of the epileptiform activity was demonstrated by comparing a baseline image collected before stimulation to those obtained immediately after stimulation. The intensity and spread of the optical changes were much less following the second stimulation (shortest least intense after-discharge episode) than after the fourth stimulation (longest most intense after-discharge episode).

When the optical changes were below baseline, the surface EEG recordings did not identify epileptiform activity (n=3 patients). At site 3, the optical changes after stimulation were below baseline. However, during the fourth stimulation, the epileptiform activity spread into the area of site 3 and the optical signal did not go below baseline until later. This negative optical signal likely represents inhibited neuronal populations (an epileptic inhibitory surround), decreased oxygen delivery, or blood volume shunted to activated regions.

Percentage difference images representative of various times during two of the stimulation trials described above were acquired. The three images were from stimulation trial 2, where 6 mA cortical stimulation evoked a brief period of after discharge. These were compared to three other images, which were from stimulation trial 4, of the optical changes evoked by cortical stimulation at 8 mA. Control images during rest were also compared. The peak optical changes occurring during the epileptiform after discharge activity were compared and the degree of recovery 20 seconds after the peak optical changes were observed, were compared. The magnitude of optical change were indicated by grey-scale changes. Each image mapped an area of cortex approximately 4 cm by 4 cm.

Eight percentage difference images were obtained from stimulation trial 2. Each image was integrated over a two second interval. The focal area of greatest optical change was in the center of the images indicating the region of greatest cortical activity. This region is the epileptic focus. The images included magnitude of optical change and the direction of increasing amplitude. Each image mapped an area of cortex approximately 4 cm by 4 cm.

A real-time sequence of dynamic changes of stimulation-evoked optical changes in human cortex was also obtained. Eight images were formed, where each image was for an average of 8 frames (<¼ second per image). The magnitude of optical change was indicated. Each image mapped to an area of cortex that is approximately 4 cm by 4 c m.

Thus, the methods and apparatus of the present invention can be used to map, in real time, dynamics of optical changes, and display such information to a surgeon in an informative format.

### EXAMPLE 2

Activation of sensory cortex by stimulation of a peripheral nerve was imaged using a rat model to show activation of somatosensory cortex by stimulation of a peripheral nerve in an anesthetized rat (inducing afferent sensory input by directly stimulating the sciatic nerve in the hind limb of a rat). One image was a grey-scale image of hind limb somatosensory cortex in an anesthetized rat. The magnification was sufficiently high so that individual capillaries can be distinguished. Another was an image of a percentage difference control optical image during rest. The magnitude of optical change was indicated. An additional image was a percentage difference map of the optical changes in the hind limb somatosensory cortex during stimulation of the sciatic nerve.

Thus, it was demonstrated that the method and apparatus of the present invention may be used to map functional areas of the cortex providing afferent input while the subject is anesthetized.

### EXAMPLE 3

This example illustrates the use of a contrast enhancing agent, indocyanine green, to identify and spatially localize a low grade human CNS tumor. An MRI scan was conducted before the operation. Additionally, the patient was investigated for tumor tissue using the apparatus described according to the invention and specifically described above.

The area of interest was evenly illuminated by a fiber optic light source with the radiation passing through a beam splitter, controlled by a D.C. regulated power supply (Lambda, Inc.) and passed through a 695 nm long pass filter. Images were acquired with a CCD camera (COHU 6500) fitted to the operating microscope with a specially modified cineadaptor. The cortex was stabilized with a glass footplate. Images were acquired at 30 Hz and digitized at 8 bits (512×480 pixels, using an Imaging Technology, Inc. Series 151 system, Woburn, Mass.). Geometrical transformations were applied to images to compensate for small amounts of patient motion (Wohlberg, Digital Imaging Warping, IEEE Computer Society: Los Alamitos, Calif., 1988). Subtraction of images collected following dye administration from those collected during a control state with subsequent division by the control image resulted in percentage difference maps. Raw data (i.e., no digital enhancement) were used for determining the average optical change in specified regions (average size site was 30×30 pixels or 150-250 um²). For pseudocolor images, a linear low pass filter removed high frequency noise and linear histogram transformations were applied. Noise was defined as the standard deviation of fluctuations in sequentially acquired control images as 0.003-0.009.

An averaged control image was obtained of the particular cortical surface area of interest. Indocyanine green dye was administered into a peripheral intravenous catheter as a bolus at Time 0. Lettered labels were placed upon the brain by the surgeon overlay the tumor as identified intraoperatively by ultrasound. However, tumors of this type and grade are notoriously difficult to distinguish from normal tissue once the surgical removal of the tumor has begun. Difference images were taken approximately 15 seconds after intravenous injection of dye (indocyanine green at 1 mg/kg), and 30 seconds after dye administration. The area of the tumor tissue showed the first tissue staining. Another image showed that in this low grade tumor, all tissue (both normal and abnormal) showed staining at 45 sec after dye administration. Other images were taken at one minute after dye administration and at five minutes after dye administration to show complete clearance in this low grade tumor. These data show that indocyanine green enters low grade tumor tissue faster than normal brain tissue, and may take longer to be cleared from benign tumor tissue than normal tissue. Therefore, it is possible to image even low grade tumors with this apparatus. Furthermore, it is possible to distinguish, intraoperatively, low grade tumor tissue from surrounding normal tissue.

### EXAMPLE 4

This example illustrates the image of a highly malignant CNS tumor (glioblastoma). A patient was imaged in a neurosurgical procedure as described in Example 1. A series of images were taken from the cortex of a patient with a malignant CNS tumor (glioblastoma; astrocytoma, Grade IV). One image showed a grey-scale image in which malignant brain tumor tissue was densest in the center and to the right but elsewhere was mostly normal tissue (as was shown by pathology slides available one week after surgery). A difference image was obtained at 15 seconds after intravenous injection of indocyanine green, showing the dynamics of dye perfusion in the first seconds in malignant tissue are similar to those in the first few seconds of benign tumor tissue. An image taken at 30 seconds showed that the malignant tissue is even more intense by comparison to the normal tissue. In addition, images taken at 1 minute after dye injection and 10 minutes after dye injection both showed that unlike benign tumor tissue, in malignant tumor tissue, dye was retained significantly longer, and in some cases, continued to sequester in the malignant tumor tissue over longer periods of time. Therefore, it is possible with this device to identify malignant tumor tissue, distinguish intraoperatively between normal and malignant tumor tissue, and to distinguish between the various grades of tumor (e.g., normal vs. benign vs. malignant). Thus, it is possible to not only image the location of tumor tissue, but also to grade the tumor with more malignant tumors retaining dye for a longer period of time than a lower grade tumor.

### EXAMPLE 5

This example illustrates that the methods and apparatus of the present invention can be used to characterize and identify tumor tissue that does not contrast enhance with traditional MRI imaging. Lack of MRI enhancement is usually typical of benign tumors. However, a proportion of non-benign tumors are not observable with present MRI imaging techniques. Images were taken from a patient whose tumor did not contrast enhance with MRI. However, optical imaging was able to identify this tumor as a non-benign type. Pathology and flow cytometry data available one week after surgery confirmed that this tumor was an anoplastic astrocytoma. A first image simply showed the gray-scale image of the area of interest and second image showed the difference image prior to dye injection. Another showed the area of interest 1 minute after intravenous dye injection, and a further image showed the area of interest 5 minutes after dye injection. It was observed that the dye was retained in this tissue for a significant time.

### EXAMPLE 6

This example illustrates a series of experiments using a rat glioma model and optical detection techniques through an intact skull to investigate whether the inventive method and inventive device could function in to image tumor tissue through an intact skull and through intact skin prior to or after surgery. Far red wavelengths of emr are known to penetrate through bone and skin. Imaging of tumor tissue was attempted through the intact skull of the rat. The extent of tumor identified was not as accurate as with the cortex exposed, however, the area lying beneath the skull with tumor tissue was easily identified, localized and continued to concentrate dye after several minutes. Initially, after dye injection, the area of the tumor demonstrated a much larger signal than the normal brain of the contralateral hemisphere. One minute after dye injection, the dye had been cleared from the normal brain and the only residual signal remained in tumor tissue and the sagital/transverse sinuses.

An image was taken for a grey-scale image of the cranial surface of a rat. The sagital suture runs down the center of the image. Tumor cells had been injected into the left side some days earlier so that this animal had developed a glioma on the left hemisphere of its brain. The right hemisphere was normal. A site 1 laid over the suspect region of brain tumor, and a site 2 laid over normal tissue. A difference image was acquired at 1 second after indocyanine green dye had been intravenously injected into the animal. The region containing tumor tissue becomes immediately visible through the intact cranium. A subsequent image showed that at 5 seconds after dye injection, the dye can be seen to profuse through both normal and tumor tissue. An image obtained at 1 minute after dye injection showed that the normal tissue had cleared the dye, but dye was still retained in the tumor region. A concentration of dye in the center of this difference image was observed and depicted dye circulating in the sagital sinus.

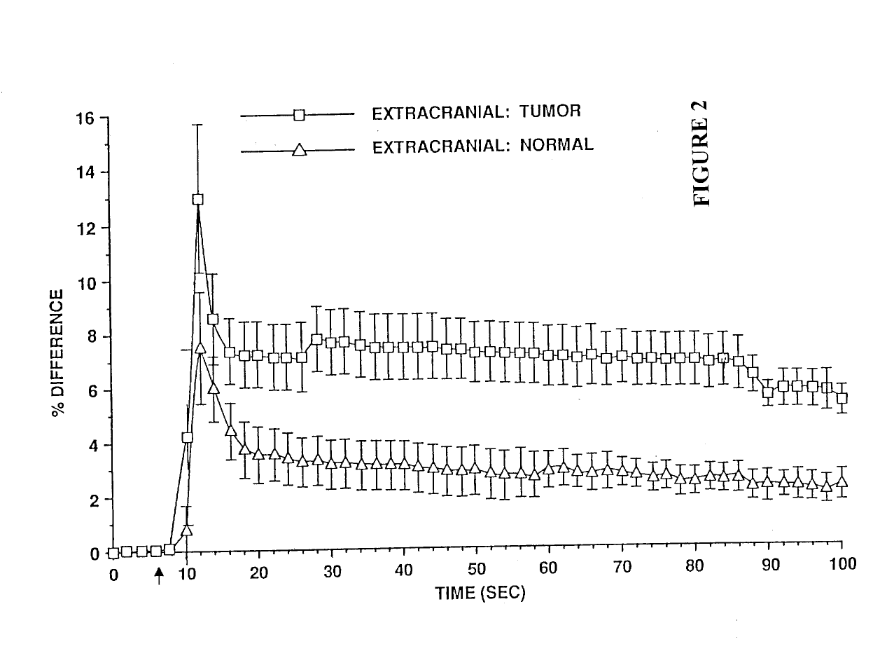

>>The time course of optical changes imaged through the cranium from ten runs in four animals are shown in FIG. 2. The optical changes were determined by the average optical change in a site placed directly over the tumor and over the normal hemisphere. The increase in absorption is a function of the concentration of dye in the tissue at a particular time. The graph labeled “extracranial tumor” is a plot of the dynamics of the absorption changes from the tumor area. The graph labeled “extracranial: normal” is a plot of the dynamics of the absorption change from the non-tumor area. The peak optical changes for the tumor imaged through the cranium were 13.1±3.9% and this was significantly greater compared to normal brain of 7.8±2.3% (p<0.01). The plateau phase 60 seconds after dye injection was also significantly greater in tumor tissue (40.5±9.6%) compared to normal brain (3.1±0.7%) (p<0.01).

These studies further demonstrate that areas of interest, and in particular, neuronal activity may be imaged through intact tissues, such as bone, dura, muscle, connective tissue and the like. As previously described, this example illustrates identification of a brain tumor through an intact cranium.

### EXAMPLE 7

This example illustrates a series of experiments using a rat glioma model intraoperatively to investigate whether the inventive method and inventive device could function in an operating room setting to provide real time information to the surgeon regarding resection of all tumor tissue. The rat glioma model is a standard predictive model and was used to delineate dye uptake, clearance and overall parameters of optical imaging that result in the best images. The advantages of this model are the ability to consistently get reproducible tumors for imaging studies and to be able to rescect tumor under an operating microscope and still find residual tumor with the inventive optical imaging. A disadvantage of this model is the more sarcoma-like appearance of the tumor and a lesser degree of vascularity compared to human gliomas.

Briefly, the rat glioma model uses an ethylnitrosourea-induced F-344 rat tumor line developed from a clonal population of a spinal malignant astrocytoma. This tumor is similar to human astrocytomas microscopically and in vivo, because both have stellate-shaped cells in the brain parenchyma and both have introcytoplasmic filaments 80-100 mm in diameter as seen by scanning electron microscopy. The glioma cells were maintained in Weymouth's medium supplemented with 10% fetal calf serum. Viable cells (5×10⁴) were trypsinized from a monolayer culture and implanted stereotaxically into the right cerebral hemisphere of 30 syngeneic female rats, each weighing 140-160 g. The stereotaxic coordinates for right frontal lobe implantation were 4.5 mm anterior to the frontal zero plane, 3 mm right from the midline and 6 mm deep. The rats were anesthetized for implantation. The heads were shaved and scalps opened, and a 1 mm burr hole made at the appropriate coordinates. The cells were injected through a 27 gauge needle, the needle left in place for 30 sec post injection and the hole was covered with bone wax: The scalp was sutured and the animals observed for 3-4 hrs until they returned to normal activity and feeding. The animals were used !I 0-14 days after tumor implantation. In this model, animals begin to show clinical symptoms from the tumor by 16-19 days, such as decreased activity and feeding, hemiparesis and eventually succumb between 19-27 days from mass effects due to tumor expansion.

Fourteen animals underwent complete study, including imaging before and after resection of the tumor. For study, the animals were anesthetized with 2% isoflurane, and the femoral vein canulated for administration of the dye. Anesthesia was maintained with a chloralsoe (50 mg/kg administered ip) and urethane (160 mg/kg administered ip). The animals were placed in a stereotaxic holder. Imaging studies were then carried out before or after removal of the cranium. The tumor typically occupied the anterior one half to two thirds of the right hemisphere exposure. The compressed brain without any tumor infiltration was defined as the tumor surround to separate it from the normal hemisphere on the contralateral side. Indocyanine green was used as the intravenous dye, although other contrasting agent may be employed. No dye was found in the cerebrospinal fluid after administration.

The cortical surface was first imaged, and then an operating microscope was used to attempt gross total removal of the tumor. Sites were then chosen for biopsy based on optical imaging results and later analyzed histologically. The biopsy specimens were fixed in 10% paraformaldehyde, Nissl stained and mounted. All specimens were read blindly and labeled either positive or negative for tumor. These data were correlated to the optical imaging results to identify residual tumor and statistical analysis (Chi square or student t-test) performed to determine the significance of the results.

The imaging apparatus used was as follows. Light was from a tungsten-halogen bulb regulated by a D.C. power supply, passed through a longpass filter (690 nm), and through a right angled prism reflected through a 50 or 100 mm objective lens onto the cortical surface. The reflected light was collected by the same objective lens and focused by a projection lens onto the surface of a CCD camera (COHU 6300). The imaging apparatus was attached to the stereotaxic frame which was rigidly fixed to a vibration isolation table. Specially designed automatic warping algorithms were designed to compensate for small amounts of movement. Images (512×480 pixels) were acquired at 30 Hz and digitized at 8 bits (256 grey levels). Every 2 sec, a single image comprising 30 averaged frames was collected (1 sec) and then stored (1 sec). Control images were collected prior to intravenous injection of the indocyanine green dye at a dose of 1 mg/kg and then for 2 min after dye injection. The dye injection was made over a 1 sec period while the last control image was being stored. A period of 20 min was allowed between dye injections to allow optical images to return to baseline. The initial control images of each trial were subtracted from each other to insure that the baseline starting point of each trial was equivalent.

A single control image was chosen and then subtracted from each of the controls (4-6 images) and each of the post-dye injection images. The resultant image was divided by the original control image and multiplied by 100 to give a composite percentage difference for the entire sequence before and after dye injection. The optical change that occurred between separate control images was 0.2-0.7%, whereas the peak changes resulting from dye injection were in the range of 5-40%. The control percentage difference images are acquired. The spatial resolution of an individual pixel in the image ranged from 13.5×11.7 mm² to 27×25.4 mm². Sites measuring from 15-30 pixels per side were drawn on the images. The average percentage change in the individual sites was calculated and used to demonstrate graphically the optical changes over time in the different types of tissue.

Imaging studies were performed on fourteen animals. The time course of dye perfusion through the tissue had a dynamic aspect. Optical imaging of indocyanine green dye perfusion at a dose of 1 mg/kg in 16 separate runs from a cortical surface in 9 different animals demonstrated the dynamic nature of the optical changes. In all rat imaging examples presented herein, each image covers an area no greater than approximately 1 cm×1 cm.

A sequence of images were acquired to illustrate the dynamic differences of dye absorption changes between tumor and non-tumor tissue. A first image showed a grey-scale image of the area of interest. This was the same animal described in Example 6, however the cranium had now been removed so as to expose the left hemisphere containing the glioma, and the right hemisphere containing normal tissue. A site 1 overlaid the tumor, site 2 the tumor-surround, and site 3 overlaid normal tissue. A difference image was taken of the area of interested 1 second after 1 mg/kg of indocyanine green had been intravenously injected into the animal. During this initial time, the tumor tissue is the first to show a measurable optical change indicating the uptake of dye occurs first in the tumor tissue. The grey-scale bar indicate the relative magnitude of the optical changes in the sequence of difference images. Further difference images of the area of interest 4 seconds and 30 seconds respectively after dye injection were obtained. At these intermediate stages dye appeared to collect in both normal and tumor tissue. Subsequent difference images of the area of interest at 1 minute and 5 minutes respectively after injection of dye clearly showed that dye was still collecting in tumor tissue even thought it was being cleared from normal tissue.

The optical signals began to change within the first 2-3 seconds after dye injection and peak 6 seconds after injection in all three areas, tumor tissue, tumor surround and normal brain. However, the three different tissue types were differentiated by the rate of rise over the first four seconds, the peak optical change reached, and the eventual plateau that occurs after the first 30 seconds. The tumor tissue had a significantly different peak percentage difference change (40.5±9.6%) than either the tumor surround (16.4±6.8%) or the normal brain (9.7±4.7%).

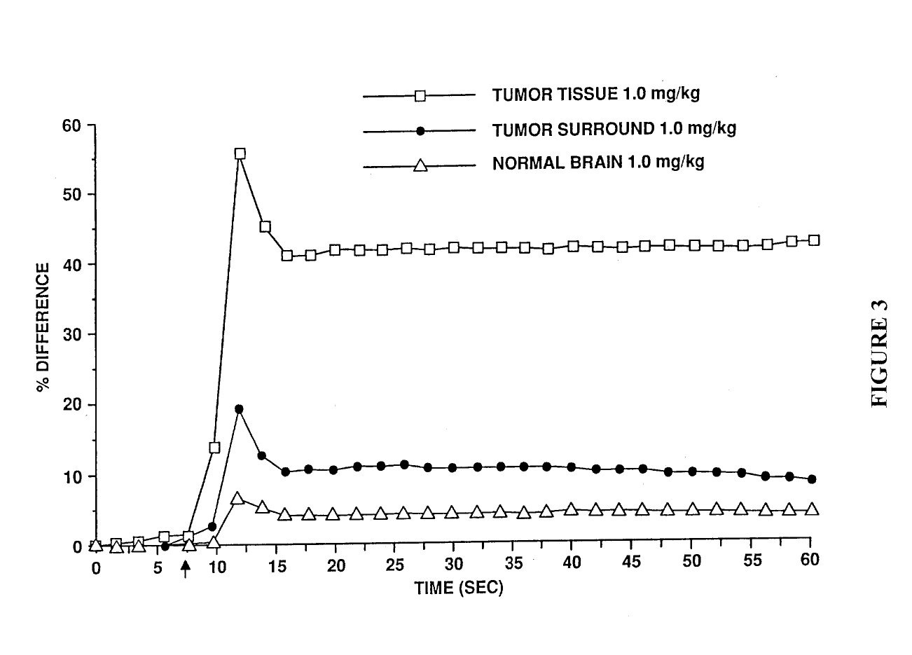

FIG. 3 is a plot of an average of the percentage change in optical properties over time averaged over the spatial areas indicated by sites 1, 2, and 3. The change in optical property is a function of the concentration of dye in the tissue at a particular time. The graphs labeled “tumor tissue,” “tumor surround,” and “normal brain” are plots of the change in optical properties over time within sites 1, 2, and 3, respectively. These data, as well as the previously described data, show that the inventive method and device is able to distinguish not only tumor from non-tumor tissue, but also tumor-surround areas which contain varying densities of tumor versus normal cells.

Since the peak optical change was always reached 4-6 seconds after dye injection, there was also a significantly faster rate of optical change in the tumor tissue compared to the tumor surround or the normal brain. A more rapid onset of dye perfusion into the tumor tissue was displayed as a faster time course. The tumor tissue had a more rapid and greater rise time than either the tumor surround or normal brain (p<0.01).

In 13 of 14 animals there was a prolonged increase (>2 min) in the optical signal in the tumor after the normal and tumor surround tissue had returned to baseline. Finally, even the normal and tumor surround tissue were significantly different in dye uptake (rise time: normal 2.4%/sec; tumor surround 4.0%/sec). Therefore, the dynamic features of dye uptake and clearance are critical for determining the type of tissue involved in imaging resection margins.

The rat glioma model also provided an opportunity to image resection margins once all visible tumor had been removed. A higher magnification image of the left hemisphere tumor margin of the animal after the tumor had been resected was observed. A site 1 overlaid areas that contained small traces of residual tumor cells, and a site 2 overlaid areas that contained only normal tissue. The gray-scale bar indicated the magnitude of optical change in the difference images. Difference images of the tumor margin were taken at 4, 30, and 60 seconds after intravenous dye injection, respectively. Minute biopsies were taken from areas that showed preferred dye containment and from areas from which the dye cleared rapidly. These biopsies were analyzed blindly and later correlated to the location from which the biopsies were taken. Those biopsies taken from areas which cleared dye were shown to contain only normal cells, whereas biopsies taken from areas which sequestered dye were shown to contain tumor cells.

The more rapid rate of rise seen in cortical surface imaging was still present for the resection margins that were positive for tumor compared to normal brain. Again, significant differences between the tumor and the normal brain existed for the rate of rise, peak optical change, and plateau 60 seconds after dye injection (all p<0.01). This experimental data demonstrates that the inventive method and device can be used in combination with multiple injections of dye for repeated application throughout a tumor resection surgery (in this case, 4 separate injections of dye were given). Furthermore, extremely small islands of residual tumor can be mapped within the tumor margins.

Sensitivity and specificity of optical imaging was determined for 34 samples (n=12 animals). Of 15 biopsy sites deemed negative for tumor by optical imaging, 14 of the 15 were clear of tumor by histological analysis (sensitivity 93%). Most of the specimens that were negative for tumor were taken from the posterior wall of the tumor resection cavity or the depth of the cavity (where the hippocampus or denate gyrus were frequently biopsied). Of 19 biopsy sites deemed positive for tumor by optical imaging, 17 of the biopsy specimens were read as positive for tumor (specificity 89.5%). The two sites that were negative for tumor on histology but positive for tumor by optical imaging had increased cellularity but were deemed negative for tumor because there was no focus of tumor tissue present. The overall significance of these results are p<0.001.

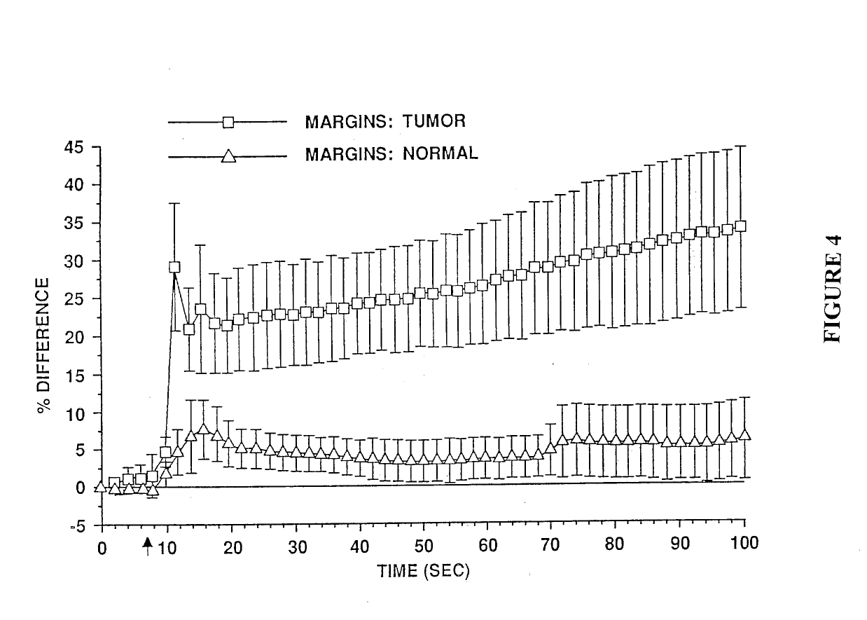

FIG. 4 shows changes in optical properties due to dye uptake and clearance in tumor vs. non-tumor tissue. Specifically, this is a plot of an average of the percentage change in optical properties over time averaged over the spatial areas indicated by sites 1 and 2. The increase in absorption is a function of the concentration of dye in the tissue at a particular time. The graphs labeled “margins: tumor” and “margins: normal”, are plots of the changes in optical properties over time within sites 1 and 2, respectively. These data show that the inventive device and method are able to distinguish tumor from non-tumor tissue within tumor margins with extremely high spatial and temporal resolution.

### EXAMPLE 8

The invention provides a method for monitoring these changes of flow within individual blood vessels. A hind limb somatosensory cortex was tested in an anesthetized rat to demonstrate measurement of blood flow rates within vessels of diameters as small as 2 mm in accordance with the present invention, thereby providing spatial resolution that is far greater than conventionally available. An image was taken to show a grey-scale image mapping an area of a rat cortex that is approximately 1 mm by 1 mm showing exemplary data acquisition sites 1, 2, and 3 encompassing an arterial, a venule, and cortical tissue, respectively. The image mapping was formed with a CCD camera (COHU 6500) that is fitted to an operating microscope and acquires image frames of 512×480 pixels at 30 Hz. The image frames are preferably digitized at 8 bits using a Series 151 system from Imaging Technology Inc. of Woburn, Mass. The 2 micron image resolution represents the resolution of individual pixels within the 1 mm by 1 mm mapping, which allows individual capillaries to be distinguished. It will be appreciated that higher spatial resolutions can be achieved with even greater microscopic magnification.

Differences in blood flow rate correspond to differences in emr absorption and, therefore, differences in the light received by the CCD camera. For example, increased flow of oxygenated blood corresponds to an increase in the ratio of oxyhemoglobin to deoxyhemoglobin, which would appear brighter (or darker) if the emr detected by the CCD camera is filtered to pass red (or green) light. Similarly, increased flow of deoxygenated blood corresponds to a decrease in the ratio of oxyhemoglobin to deoxyhemoglobin, which would appear darker (or brighter) if the emr detected by the CCD camera is filtered to pass red (or green) light. Moreover, the ability to measure blood flow changes over periods of 0.5 second or less provides a temporal resolution for blood flow measurement in small vessels that contrasts very favorably with conventional techniques that are capable of detecting blood flow changes only over periods of several minutes or more.

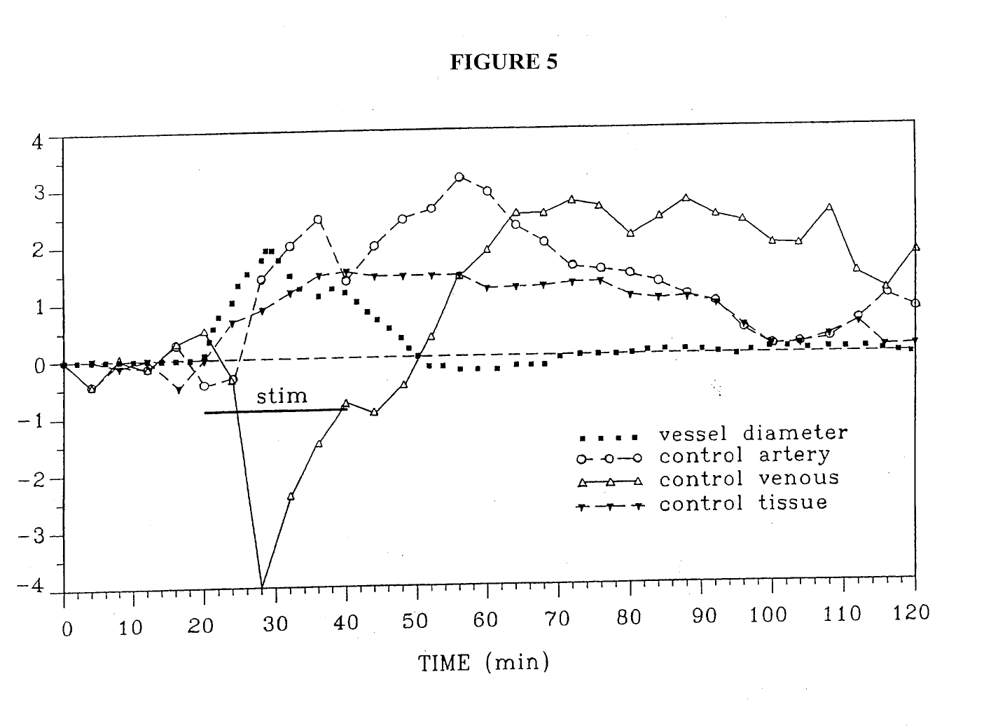

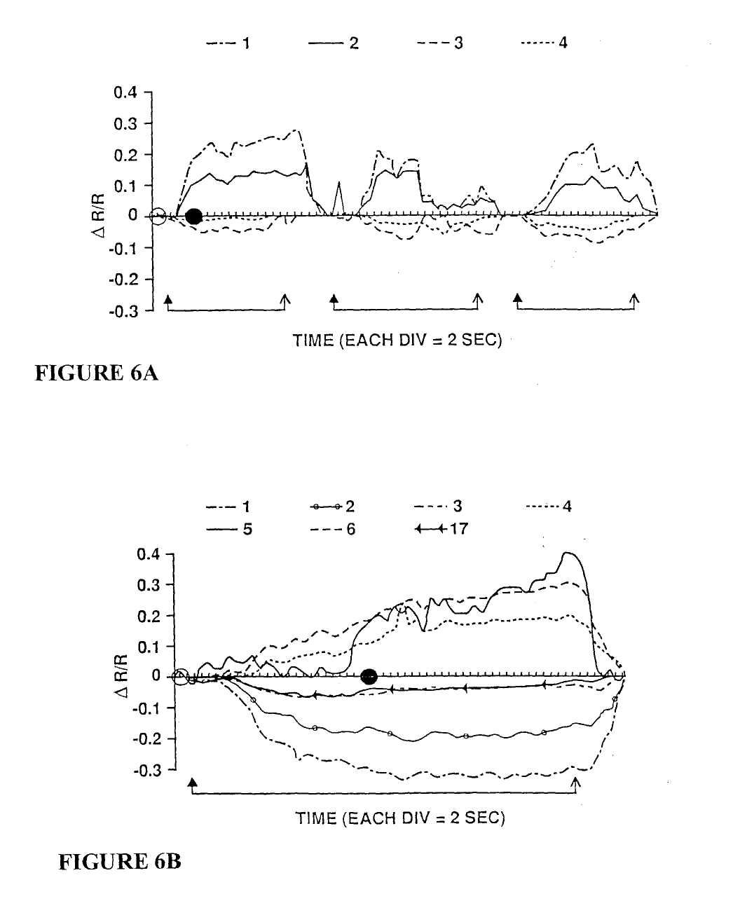

FIG. 5 shows plots of percentage change of emr absorption per second in the spatial regions of sites 1, 2, and 3 and a plot of corresponding morphological measurements of the venule in the spatial region of site 2. The change in emr absorption is measured during activation of somatosensory cortex in an anesthetized rat by direct stimulation of the sciatic nerve in the hind limb of the rat relative to a baseline level of somatosensory cortical activity prior to stimulation. Each data point corresponds to an average of pixel values within the corresponding sample site obtained from 16 frames over about 1/2 second at intervals of one second.

FIG. 5 shows positive-going changes in emr absorption corresponding to increased flow of oxygenated blood in the arterial encompassed by site 1. The plot represents a period that includes a baseline level of cortical activity prior to stimulation of the sciatic nerve, stimulation of the nerve, and a subsequent recovery period. FIG. 5 also shows corresponding negative-going changes in emr absorption corresponding to increased flow of deoxygenated blood in the venule encompassed by site. These plots demonstrate the effectiveness of measuring positive- and negative-going emr absorption representing blood flow at high spatial and temporal resolutions in accordance with the present invention.

FIG. 5 also shows corresponding morphological measurements of the diameter of the venule in the spatial region of site 2. The morphological measurements correspond to widths of the venule measure from video images. As is known in the art, vessel diameter relates to blood rates by a power of three. This plot serves as a control of the plotted blood flow rates measured in accordance with the present invention. It will be appreciated, however, that the blood flow rates measured in accordance with the present invention have significantly higher resolution that the relatively simple morphological measurements.

FIG. 5 further shows changes in emr absorption in the somatosensory cortical tissue encompassed by site 3. These emr absorption changes may relate to the plotted blood flow changes, as well as other intrinsic tissue characteristics. This demonstrates how the high spatial and temporal resolutions with which emr absorption can be measured in accordance with the present invention can allow determination of whether changes in tissue characteristics correlate to blood flow rates or other intrinsic factors.

A sequence of contrast enhanced images were obtained showing dynamic changes of optical signals corresponding to blood flows plotted in FIG. 5. One image represented a control image corresponding to baseline cortical activity prior to stimulation of the rat sciatic nerve. Other images represented successive difference images corresponding to positive-going changes in emr absorption following stimulation of the rat sciatic nerve. Still other images represented successive difference images corresponding to positive-going changes in emr absorption of cortical tissue during the recovery following stimulation of the rat sciatic nerve. In these figures, stimulation causes arterials to show increased red brightness, which corresponds to increased flow of oxygenated blood. Venules appear darker in response to stimulation, corresponding to increased flow of deoxygenated blood. The images during the recovery period showed blood flow rates returning to baseline amounts.

A pair of images were formed by converse subtractive calculations to show thee opposite changes of optical signals corresponding to arterials and venules. One image was a difference image selected to show with increased red brightness arterials with increased flow of oxygenated blood. Another image was a difference image showed increased brightness venules with increased flow of deoxygenated blood. Still, another image showed that converse difference images, which can be rendered individually (as shown) or together, can be used to illustrate opposing emr absorption changes relating to arterial/venule or oxygenated/deoxygenated blood flow.

### EXAMPLE 9

Identification of cortical areas of neuronal inhibition was tested by using a human cortex just anterior to face-motor cortex and employing one recording (R) and two stimulating electrodes (S). Four images were taken, each corresponds to an average of approximately 60 frames which were acquired to 30 Hz over a period of about 2 seconds. Each image mapped to an area of cortex that was approximately 4 cm by 4 cm. The cortex was illuminated with emr of wavelengths greater than about 690 nm and images were obtained representing changes in absorption of emr over different periods. Normally areas of increased neuronal activity (or intrinsic signals) result in an increase of emr absorption capacity of neuronal tissue (i.e., the tissue appears darker if visible light is used for emr illumination). Similarly, a decrease in neuronal activity (or intrinsic signals) results in a decrease of emr absorption capacity of the tissue (i.e., the tissue appears brighter).

One image was a spatial map of baseline cortical activity prior to application of stimulating current for inducing epileptiform activity. The baseline cortical activity corresponds to period A in the EEG recording of surface electrical signals received by a recording electrode (R).

Another image was a spatial map of cortical activity during 6 mA stimulation at stimulating electrodes (S) and the resulting epileptiform after discharge activity. This cortical activity corresponds to period B in the EEG recording of surface electrical signals received by a recording electrode (R). This image showed an area that encompassed recording electrode (R) and corresponds to increasing (positive-going) cortical (neuronal) activity and significantly elevated signal levels in the EEG recording. However, the elevated signal levels over period B in the EEG recording mask large surrounding darker region corresponding to decreasing (negative-going) cortical (neuronal) activity.

Another image was a spatial map of cortical activity during an apparent quiescent period following the epileptiform after discharge activity induced by stimulation at stimulating electrodes (S). This cortical activity corresponded to period C in the EEG recording of surface electrical signals received by a recording electrode (R). The apparently quiescent nature of period C is based upon the conventional interpretation of the decreased signal levels in the EEG recording over this period. This image showed a region that encompassed recording electrode (R) and corresponded to decreasing (negative-going) cortical (neuronal) activity. However, the decreased signal levels over period C in the EEG recording masked a significant darker gray region, extending between stimulating electrodes (S) but not to recording electrode (R), corresponding to increasing (positive-going) cortical (neuronal) activity. As a result, the decreased or quiescent signal levels over period C in the EEG recording masked a significant lighter gray region corresponding to increasing (positive-going) cortical (neuronal) activity.

Still a fourth image was a spatial map of cortical activity during a period following a quiescent period. This cortical activity corresponded to period D in the EEG recording of surface electrical signals received by a recording electrode (R). This image showed a region of mixed lighter and darker gray subregions that encompassed recording electrode (R) and corresponds to increasing (positive-going) cortical (neuronal) activity and signal levels in the EEG recording that were elevated in comparison to the quiescent characteristics of period C. However, the elevated signal levels over period D in the EEG recording masked large adjacent red region corresponding to increasing (positive-going) cortical (neuronal) activity.

Cortical areas of neuronal inhibition may be identified by subtractive processing of difference images. For example, one image may be a subsequent averaged image and another image may be an averaged control image (e.g., a spatial map of baseline cortical activity). Conventionally, when a pixel in one image is subtracted from a pixel in another image and a negative value results, this value is treated as zero. Hence, difference images cannot account for areas of inhibition. This is a disadvantage of conventional EEG techniques, as well as conventional optical imaging, magnetic resonance imaging, and positron emission tomography.

However, the present invention provides a method for identifying both negative and positive neuronal activity (intrinsic signals) by the method comprising: (a) subtracting one image A (a subsequent averaged image) from another image B (an averaged control image) to create a first difference image, whereby all negative pixel values are zero; and (b) subtracting image B from image A to create a second difference image whereby all negative pixel values are zero; and adding the first and second difference images to create a “sum difference image”. The sum difference image shows areas of increased activity and show areas of less activity or inhibition. The four spatial maps were generated in this manner. Alternatively, one can overlay the first difference image on the second difference image. Either method provides an image of increased neuronal activity and decreased neuronal activity.

The high resolution of the spatial maps, together with identification of areas of both increased and decreased neuronal activity, can be used by a neurosurgeon intraoperatively to identify precisely areas in the brain affected by epileptiform after discharge activity. This allows neurosurgery to be performed with minimal damage to other cortical areas.

### EXAMPLE 10

Sprague-Dawley rats (male and female; 25 to 35 days old) were prepared as described in Aghajanian, A. K. and Rasmussen, K., Synapse 31:331, 1989; and Buckmaster, P. S., Strowbridge, B. W., Schwartzdroin, P. A., J. Neurophysiol. 70:1281, 1993. In most hippocampal slice experiments, simultaneous extracellular field electrode recordings were obtained from CA1 and CA3 areas. For stimulation-evoked after discharge (13 slices, 8 animals), the concentration of Mg²⁺ in the bathing medium was reduced to 0.9 mM. A bipolar tungsten stimulating electrode was placed on the Schaffer collaterals to evoke synaptically driven field responses in CA1; stimuli consisted to 100 to 300-μs-duration pulses at an intensity of four times population-spike threshold. After discharges were evoked by a 2-strain of such stimuli delivered at 60 Hz. Spontaneous interictal-like bursts were observed in slices treated with the following modifications or additions to the bathing medium: 10 mM K⁺ (6 slices; 4 animals; average, 81 bursts/min), 200 to 300 μM 4-AP (4 slices; 2 animals; average, 33 bursts/min), 50 to 100 μg M bicuculine (4 slices; 3 animals; average, 14 bursts/min), 0 mM Mg²⁺ [(1 hour of perfusion) 3 slices; 2 animals; average, 20 bursts/min; (3 hours of perfusion) 2 slices, 2 animals)], 0 mM Ca²⁺/6 mM KCl and 2 mM EGTA (four slices, three animals). In all treatments, perfusion with furosemide-containing medium was begun after a consistent level of bursting had been established.

For imaging of intrinsic optical signals, the tissue was illuminated with a beam of white light (tungsten filament light and lens system; Dedotec USA, Lodi, N.J.) directed through the microscope condenser. The light was controlled and regulated (power supply: Lambda Electronics, Melville, N.Y.) to minimize fluctuations and filtered (695 nm long-pass) so that the slice was transilluminated with long wavelengths (red). Image frames were acquired with a charge-coupled device camera (Dage-MTI) at 30 Hz and were digitized at 8 bits with a spatial resolution of 512 by 480 pixels by means of an Imaging Technology Series 151 imaging system; gains and offsets of the camera-control site and the analog-to-digital board were adjusted to optimize the sensitivity of the system. Imaging hardware was controlled by a 486-PC-compatible computer running software written by D. Hochman and developed with commercially available software tools (Microsoft's C/C++ Compiler and Imaging Technology's ITEX library). To increase signal-to-noise ratio, an averaged image was composed from 16 individual image-frames, integrated over 0.5 s and averaged together. An experimental series typically involved the continuous acquisition of a series of averaged images over a several minute time period; at least 10 of these averaged images were acquired as control images before stimulation. Pseudocolored images were calculated by subtracting the first control image from subsequently acquired images and assigned a color lookup table to the pixel values. For these images, usually a linear low-pass filter was used to remove high-frequency noise and a linear-histogram stretch was used to map the pixel values over the dynamic range of the system. All operations on these images were linear so that quantitative information was preserved.

Several images were obtained to show the effect of the agent furosemide on stimulation evoked after discharge activity in a hippocampal tissue slice comparing the field response, measurements at an extracellular electrode, and images highlighting changes in optical properties.

One illustrated that two seconds of electrical stimulation at 60 Hz elicited after discharge activity. Another image showed a typical after discharge episode recorded by the extracellular electrode, with the horizontal arrow indicating the baseline. Still another image showed a map of the peak change in optical transmission through the tissue evoked by Schaffer collateral stimulation. A grey-scale bar was used to indicate increasing magnitude of activity-evoked optical changes from the bottom to the top of the bar. A region of maximum optical change corresponds to the apical and basal dendritic regions of CA1 on either side of the stimulating electrode. Another series of images were used to illustrate responses to electrical stimulation following 20 minutes of perfusion with medium containing 2.5 mM furosemide. Both the electrical after discharge activity and the stimulation-evoked optical changes were blocked. However, there was a hyperexcitable field response (multiple population spikes) to the test pulse. Still another set of images were taken to illustrate that restoration of the initial response pattern was seen following 45 minutes of perfusion with normal bathing medium.

A set of images were obtained to illustrate an enlarged grey-scale image of an acute rat hippocampal tissue slice, observed using a CCD camera attached to a Zeiss upright microscope. These images illustrated enlarged, contrast-enhanced images acquired as described above. One image illustrated an enlarged, contrast-enhanced image acquired as described above during the peak optical change induced by electrical stimulation, with an enlarged color bar, the arrow on the color bar indicating increasing magnitude of activity-evoked optical changes. An image illustrated the peak optical change during electrical stimulation when no epileptic activity was induced. Another image illustrated the peak optical change during electrical stimulation that resulted in epileptiform activity. A larger area of increased magnitude of changes in optical properties is observed during epileptiform activity. Another image illustrated the peak optical change during electrical stimulation following treatment with furosemide, which blocks the epileptiform activity and the intrinsic optical signal.

### EXAMPLE 11

This example illustrates one technique for setting the CCD to optimize the apparatus to be able to detect signal with maximum sensitivity across a full dynamic range. The CPU should be programmed with software having the following features: (1) an output-analog signal, values of the image are close to saturating on the bright end (i.e., close to 225) are displayed as a distinct color, such as red; (2) values that are close to the dark end (i.e., are close to zero) are also displayed as a distinct color, such as blue. The following procedure is an example of an adjustment of the CCD camera.

1. With the gain and black-level on a camera-control box (CCB) initially set to 0, increase the emr intensity until the video signal is just saturating on the bright-end (i.e., some values in the output-analog signal can be seen to be close to 255).

2. Increase the black-level on the CCB until the output image can be seen to be saturating on the dark end (i.e., some values in the output analog image can be seen to be close to 0).

3. Increase the gain on the CCB until some values of the output analog image can be seen to be saturating on the high end.

4. Iterate steps (2) and (3) until either: (a) the gain is set to its maximum possible value; (b) the black-level is set to its maximum possible value; or (c) the image is maximally enhanced across is full dynamic range (that is, no further adjustments of CCB gain, black-level or emr source will improve the image).

5. If in Step 4: (a) the gain is set to its maximum level; or (b) the black-level is set to its maximum level, but the output image is still not maximally enhanced, then in the case of (a), decrease the setting on the CCB gain slightly, increase the emr source intensity until just saturating the bright end, and return to Step 2. In the case of (b), decrease the setting of the black-level slightly, decrease the emr intensity, and return to Step 3.

### EXAMPLE 12

This example illustrates various methods for enhancing images obtained from or intrinsic signal difference images using multiple wavelength and/or laser illumination, and a method for extracting 3-D information using multiple wavelengths. We expose a region of cortex in an anesthetized rat. First, illuminating with white light from a tungsten filament lamp, we acquire a sequence of difference images prior to, during, and following electrical stimulation of this region of cortex with bipolar stimulating electrodes. Next, we acquire second and third difference image sequences, following the identical procedure as we did for the first sequence, except that in the second sequence, the cortex is illuminated with 690 nm and in the third sequence 510 nm light. The change in wavelengths is accomplished by placing 690±10 nm interference filter or a 510±10 nm interference filter between the light source and the brain.

We compute the contrast-enhanced image by first ratioing a control 690 nm image with a control 510 nm image. Second, we ratio a 690 nm image during stimulation with the corresponding 510 nm image. We then combine the ratio images to compute the percentage difference image. In this manner, the noise has been significantly reduced, hence the signal/noise ratio has been significantly increased.

Next, we show how to extract depth information from the multiple wavelength images that we have acquired. Longer wavelength light penetrates to a greater depth through the cortex, and shorter wavelength light to a lesser extent. Hence, the 690 nm image as penetrated cortex to x mm, and the 510 nm image to y mm where x<y.

We subtract the 610 nm image from the 510 nm image, showing an “optical wedge” containing information from a depth of (x−y) mm to x mm within the cortical tissue. By using a series of other interference filters, we acquire a sequence of images containing information from many different depths of the cortex. It is possible to acquire 3-dimensional information.

Next, exposing tumor tissue in a rat in which we have induced tumor growth, we repeat all of the above experiments showing that in a like manner, we can improve signal/noise and extract 3-dimensional information in tumor tissue. However, instead of stimulating the tissue electrically, we inject the dyes indocyanine green or Evans blue.

Finally, we repeat the above experiments by illuminating the cortex at several different wavelengths with a dye-tunable laser (a coherent source) instead of with the non-coherent tungsten filament lamp. With the laser (or any coherent source) we have the additional advantage in that we can separate out the components of the signal due to changes in reflection or scattering. By illuminating the cortex with the laser directly parallel to the camera (both of which are perpendicular to the brain), we are imaging reflected light only. By moving the laser at an angle 0 to the camera, we are measuring changes due to scattering alone at this particular angle.

### EXAMPLE 13

In the case of tissue detection in a human subject, it is necessary to compensate for the motion of the subject which may occur between the acquisitions of consecutive images. For many types of images, it is possible to compensate by a geometrical compensation which transforms the image by translation in the x-y plane. In order for an algorithm such as this to be feasible, it must be computationally efficient (preferably implementable in integer arithmetic), memory efficient, and robust with respect to changes in ambient light.

One possible method would be to translate an image by 0 through k number of pixels in every possible direction with respect to the control image. For each of the (2*k+1)*(2k+1) translations, make a subtraction image and calculate some metric to estimate the closeness to the control image. An example of such a metric would be the variance of the subtraction image. The drawback of this method is that it is not efficient since for each of (2*k+1)*(2k+1) subtraction images, we would need to calculate the variance over 512*512 pixels.

An efficient improvement of this algorithm is to estimate the variance of the subtraction images by randomly selecting some small number of areas of interest (for example, 9 areas of interest), each area consisting of a small number of pixels (say 8×8) from the image that one wishes to translate with respect to the control image. Also, choose some search depth (for example, 10 pixels) over which to translate these small areas of interest with respect to their corresponding areas of interest in the control image. After translation in all possible directions for 0 through 10 pixels, choose the translation which minimizes the variance over the selected areas of interest. Since all the areas of interest are the same size, division is not necessary in the calculation of the variance which is to be ordered so that the minimal variance can be selected. Hence, all calculations can be carried out in integer arithmetic. Since the areas of interest are sufficiently small, most of the data can be read into the host computer's RAM limiting IO to the frame buffers and increasing speed.

### EXAMPLE 14

This example illustrates optical mapping of the margins of a malignant CNS tumor. A series of images and difference images were taken of the area of interest taken after surgical removal of the tumor and when the area was thought to be free of tumor tissue. Normally, in this size of a resection margin, only a single frozen sample would be taken for pathology analysis. For the purpose of this study, five biopsies were taken from the margin to aid in correlating the histology with the map obtained by the invention. One image showed a gray-scale image of the tumor margin. A second image showed the margin with labels that the surgeon placed directly on the brain to identify where the surgeon was going to remove biopsy samples for histological analysis after difference images were acquired with the inventive device. Another image showed the difference image 1 minute after intravenous injection of dye and a further image showed the difference image 10 minutes after dye injection. These post-dye difference images reveal a number of sites that contain tumor tissue as well as areas of normal tissue. The accuracy of the optical imaging was confirmed post operatively by analysis of the biopsies. A small area on the lower right of the 10 minute post dye image indicated a possible region of tumor tissue that would not have been biopsied by the surgeon. These data show that the invention is able to identify small remnants of tumor tissue in a tumor margin after resection of a tumor. In addition, the invention could act as an aid to removing biopsies from the site of a tumor margin, thereby reducing the sampling error associated with the presently used random sampling technique.

These data show that indocyanine green enters low grade tumor tissue faster than normal brain tissue, and may take longer to be cleared from benign tumor tissue than normal tissue. Therefore, it is possible to image even low grade tumors with this apparatus. Furthermore, it is possible to distinguish low grade tumor tissue from surrounding normal tissue intraoperatively. Subsequent pathology of this tumor tissue established it as a low grade glioma.

### EXAMPLE 15

Stimulation mapping of the cortical surface was performed on awake human patients under local anesthesia to identify sensory/motor cortex and Broca's areas. The illumination source and optical detection device and processing techniques used were the same as those described above. During three “tongue wiggling” trials, images were averaged (32 frames, 1 sec) and stored every 2 seconds. A tongue wiggling trial consisted of acquiring 5-6 images during rest, then acquiring images during the 40 seconds that the patient was required to wiggle his tongue against the roof of his mouth, and then to continue acquiring images during a recovery period. The same patient was then required to engage in a “language naming” trial. A language naming trial consisted of acquiring 5-8 images during rest (control images the patient silently viewing a series of blank slides), then acquiring images during the period of time that the patient engaged in the naming paradigm (naming a series of objects presented with a slide projector every 2 seconds, selected to evoke a large response in Broca's area), and finally a series of images during the recovery period following the time when the patient ceased his naming task (again viewing blank slides while remaining silent).

Grey-scale images were obtained of an area of human cortex, with left being anterior, right-posterior, top-superior, and the Sylvan fissure on the bottom. Specific sites in the images represented sites where cortical stimulation with electrical stimulating electrodes evoked palate tingling, tongue tingling, speech arrest-Broca's areas, and no response. One image was a percentage difference control image of the cortex during rest in one of the tongue wiggling trials. Another of the images was a percentage difference map of the peak optical changes occurring during one of the tongue wiggling trials. Areas identified as tongue and palate sensory areas by cortical stimulation showed a large positive change. Suppression of baseline noise in surrounding areas indicated that, during the tongue wiggling trials, language-motor areas showed a negative-going optical signal. Still another of the images was percentage difference control image of the cortex during one of the language naming trials. A percentage difference image was also taken of the peak optical change in the cortex during the language naming task. Large positive-going signals are present in Broca's area. Negative-going signals are present in tongue and palate sensory areas.

FIGS. 6A and 6B shows the plots of the percentage change in the optical absorption of the tissue within certain sites during each of the three tongue wiggling trials and one of the language naming trials. FIG. 6A shows the plots during the three tongue wiggling trials averaged spatially within the sites 1, 2, 3, and 4. FIG. 6B shows the plots during one of the language naming trials averaged spatially within the site 1-7 and 17.

These results agree with those data reported by Lee et al. (Ann. Neurol. 20:32, 1986), who reported large electrical potentials in the sensory cortex during finger movement. The magnitude of the optical changes in the sensory cortex during tongue movement (10-30%) parallels sensory/motor cortex Studies where cerebral blood flow increases 10-30% during motor tasks (Colebatch et al., J. Neurophysiol. 65:1392, 1991). Further, utilizing Magnetic Resonance Imaging (MRI) of blood volume changes in human visual cortex during visual stimulation, investigators have demonstrated increases of up to 30% in cerebral blood volume (Belliveau, et al., Science 254:716, 1991).

Optical images were obtained from this same cortical region (i.e., area of interest) while the patient viewed blank slides and while naming objects on slides presented every two seconds. Percentage difference maps obtained during naming showed activation of the premotor area. The sites of speech arrest and palate tingling were identified by surface stimulation and demonstrate optical signals going in the opposite direction. The area of activation was clearly different from that evoked by tongue movement without speech production. The optical images of premotor cortex activation during naming were in similar locations to the cortical areas identified in PET single word processing studies (Peterson, et al., Nature 331:585, 1991; and Frith et al., J. Neuropsychologia 29:1137, 1991). The optical changes were greatest in the area of the cortex traditionally defined as Broca's area and not in areas where electrical stimulation caused speech arrest.

### EXAMPLE 16

Human cortex was imaged using the illumination source and optical detector described in Example 1. Functional mapping was conducted prior to and during imaging.

An image was taken of the cortical surface of a patient where the anatomical orientation is left-anterior, bottom-inferior, with the Sylvan fissure running along the top. After optical imaging, all cortical tissue to the left of the thick line was surgically removed. A first site and a second site were identified as essential for speech (e.g., cortical stimulation blocked ability of subject to name objects). At a third site one naming error in 3 stimulation trials was found. As the surgical removal reached a certain area, the patient's language deteriorated. Other sites were identified that had no errors while naming slides during cortical stimulation and these sites were noted as unlabeled sites in the image. An image was taken to show an overlay of a percentage difference image over the grey-scale image of the cortex acquired during a language naming trial. The magnitude of the optical change is shown by the grey-scale bar on the lower right of the image. This data demonstrates how a surgeon might use this invention intraoperatively to map language cortex and to avoid surgically removing tissue having important functional properties.

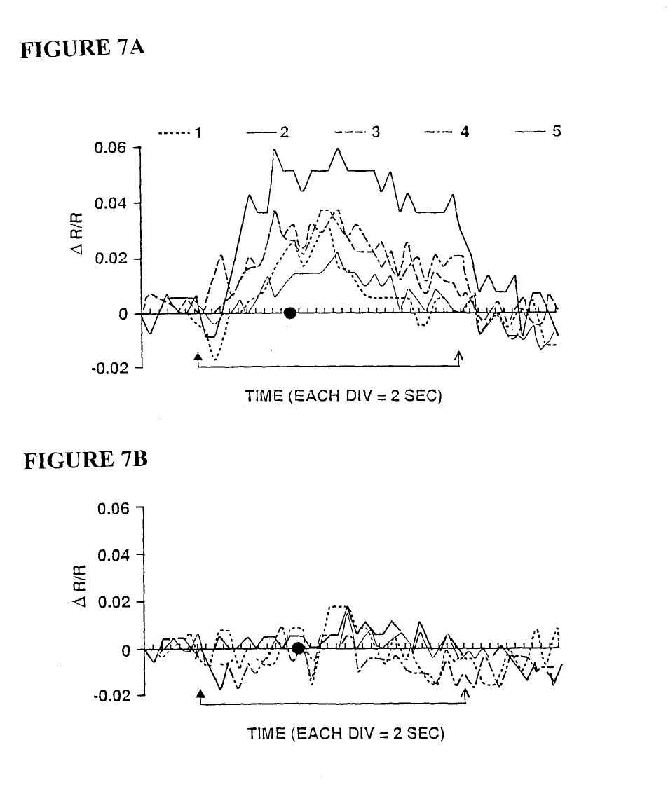

FIG. 7A shows plots of percentage change in optical absorption of tissue within the sites. The plots for sites 1 and 2 overlay essential language sites, and sites labeled 4, 5, and 6 overlay secondary language sites. Each of these five sights showed significant changes occurring while the patient was engaged in a language naming task. FIG. 7B shows percentage changes from the six unlabeled sites. There were no significant increases or decreases within these anterior sites. The data illustrated in FIGS. 7A and 7B demonstrate that optical imaging can also identify both essential and secondary language areas that must be preserved during neurosurgical procedures.

### EXAMPLE 17

Optical contrast enhancing agents may be used in connection with optical imaging techniques of the present invention. The utility of such agents may be demonstrated using hippocampal brain slice preparations. Hippocampal slices may be loaded in a chamber provided with artificial cerebral spinal fluid (“ACSF”), albumin labeled with indocyanine green (“ICG”) (approx. 2 mM) and 2% DMSO. After one hour, the tissue will be visibly stained. Because the albumin-labeled ICG collects in the extracellular space, this staining technique may be used to detect changes in neuronal activity and/or functions that are correlated to changes in the volumes of the extracellular space. Similarly, the fluorescent agent Biodipy (available from Molecular Probes, Inc., P.O. Box 22010, Eugene, Oreg. 97402) bound to albumin will collect in extracellular space and may be used as a contrast enhancing agent to detect neuronal states or changes correlated to changes in the volume of the extracellular space.

### EXAMPLE 18

This example illustrates imaging of functional regions of peripheral nerves. A rat is anesthetized and dissected to expose the sciatic nerve. Using silver electrodes we electrically stimulate the caudal end of the nerve while acquiring a first sequence of difference images. We note the extent of the spread of the intrinsic optical changes in the nerve from the point of stimulation by examining the difference imaging containing the peak optical change from the control. Next, we make a crush in the nerve at a small distance anterior to the stimulating electrodes. We acquire a second sequence of difference images and compare the corresponding difference image from this sequence to the image containing the peak optical change from the first image. We note that the intrinsic optical changes diminish abruptly at the point where the nerve was damaged.

Finally, we stimulate the nerve anteriorly to where the crush was made and after acquiring a third sequence of difference images, we again note where the intrinsic changes abruptly end. This method allows us to localize the location and extent of damaged or dysfunctional peripheral nerve tissue.

### EXAMPLE 19

This example illustrates imaging of functional regions of Cranial Nerve VIII. Cranial Nerve VIII (Vestibulococllear nerve) is exposed. Sound tones provide the auditory stimulus which eventually cause activation of this nerve. A sequence of difference images before, during, and after the appropriate auditory stimuli are applied show that intrinsic optical changes of the nerve are associated with its functional activation. Next, a small region of this nerve is damaged by crushing. A second sequence of images reveal that auditory stimulation evokes intrinsic optical changes in the nerve up to the point of damage.

### EXAMPLE 20

This example illustrates the use of methods and systems of the present invention for testing and monitoring retinal function. The retina comprises central nervous system tissue and, when activated, undergoes characteristic changes in intrinsic optical properties. Screening devices of the present invention are useful, for example, for testing the retinal function of newborns, as well as in other patient populations.

Using a probe-like instrument having one or more optical source(s) and one or more optical detector(s) mounted thereon, retinal tissue is illuminated. In this application, the illumination of retinal tissue is a stimulus of central nervous system activity. That is, impingement of emr in the visible range activates central nervous system cells in retinal tissue and produces characteristic, activity-related changes in the intrinsic optical properties of illuminated cells. In one embodiment, the area of interest from which data is acquired following illumination of retinal tissue includes both illuminated and non-illuminated tissue. In this embodiment, comparison of data points representative of intrinsic optical properties within the area of interest are compared. Retinal tissue activated by illumination exhibits different intrinsic optical properties from retinal tissue that is not illuminated and quiescent. If data acquired following illumination of retinal tissue does not show differences in intrinsic optical properties within the area of interest, the retinal tissue sample is likely to be nonfunctional. Alternatively or additionally, data acquired from an area of interest encompassing illuminated retinal tissue may be compared to one or more standard or control data sets. Although the present invention has been described in detail by way of description and examples for purposes of clarity of understanding, changes and modifications can be carried out without departing from the scope of the invention, which is intended to be limited only by the scope of the appended claims.

I claim:

1. A method for screening a patient tissue sample to spatially locate tissue having a physiological property in an area of interest, comprising:
illuminating the area of interest with an illumination source emitting electromagnetic radiation (emr) having at least one wavelength which interacts with a contrast enhancing agent wherein the area of interest is located within a patient;
administering the contrast enhancing agent to the patient;
detecting one or more optical properties of spatially resolved areas within the area of interest subsequent to administration of the contrast enhancing agent and acquiring a data set representing the one or more optical properties corresponding to each of the spatially resolved areas of the area of interest;
comparing the acquired data set representing one or more optical properties of the spatially resolved areas within the area of interest subsequent to administration of the contrast enhancing agent to a control data set, not derived from the area of interest, that represents one or more corresponding optical properties of a known tissue type or condition; and
displaying output data identifying and spatially locating the tissue having the physiological property in the area of interest based on differences between the acquired data set and the control data set.

2. The method of claim 1, wherein the tissue having a physiological property is pathological tissue.

3. The method of 1, wherein the tissue having a physiological property is dysfunctional central of peripheral nervous system tissue and further including stimulating activity in central or peripheral nervous system tissue prior to the detecting of one or more optical properties.

4. The method of claim 2, additionally comprising positioning the at least one optical source and the at least one optical detector for epi-illumination of the area of interest.

5. The method of claim 2, additionally comprising positioning the at least one optical source and the at least one optical detector for transillumination of the area of interest.

6. The method of claim 3, wherein the control data set represents one or more corresponding optical properties of spatially resolved areas empirically determined to be indicative of normal tissue of the same tissue type as the area of interest.

7. The method of claim 3, wherein the control data set represents one or more corresponding optical properties of spatially resolved areas empirically determined to be indicative of abnormal tissue.

8. The method of claim 2 further including the step of monitoring the progression or recession of pathological tissue in the area of interest.

9. A method according to claim 3 further including monitoring the healing and regeneration of central or peripheral nervous system tissue.

10. The method of claim 2, wherein the pathological tissue is cancerous tissue.

11. The method of claim 3, wherein the dysfunctional central of peripheral nervous system tissue retinal tissue and the method is for screening the retinal tissue to assess retinal function, and wherein the control data set is an empirically derived standard.

12. The method of claim 3, wherein the dysfunctional central of peripheral nervous system tissue is carpal tunnel nerve tissue and the method is for screening the carpal tunnel nerve tissue to assess carpal tunnel nerve function and wherein the control data set is an empirically derived standard.

13. The method of claim 3, wherein the dysfunctional central of peripheral nervous system tissue is spinal cord tissue and the method is for screening the spinal cord tissue to assess spinal cord function and wherein the control data set is an empirically derived standard.

14. The method of claim 1, further including assessing the safety or efficacy of a treatment agent or regimen at an area of interest targeted by the treatment agent or regimen and wherein the optical properties represented by differences in the data set and the control data set are characteristic of differences in the condition of the tissue

15. A method for detecting abnormal cortical tissue or intracranial conditions, comprising:
illuminating an area of interest including cortical tissue with electromagnetic radiation (emr) having at least one wavelength of from 450 nm to 2500 nm;
detecting one or more optical properties in spatially resolved areas within the area of interest and acquiring a data set representing the one or more optical properties of spatially resolved areas within the area of interest;
comparing the acquired data set to an empirically derived standard control data set representing one or more corresponding optical properties of spatially resolved areas having a known tissue condition; and
producing output data identifying and spatially locating differences between the acquired data set and the control data set.

16. The method of claim 15, wherein the abnormal cortical tissue or intracranial condition is selected from the group consisting of: head trauma; subdural hematoma; Alzheimer's disease; Parkinson's disease; ALS; multiple sclerosis; stroke; ischemia; hypoxia; psychiatric conditions; ethanol toxicity; epilepsy; migraine; spreading depression; depression; anxiety; bipolar disorder; schizophrenia; infection; angiogenesis; wound healing; and immune deficiencies.

17. A method according to claim 15 further including screening a tissue sample to identify and spatially locate subdural hematomas.

18. A method according to claim 15 further including screening a tissue sample to identify and spatially locate ischemic tissue.

19. A method according to claim 15 further including screening a tissue sample to identify and spatially locate hypoxic tissue.

20. A method according to claim 15 further including monitoring an abnormal cortical or intracranial condition.

21. A method for in situ screening of a patient tissue sample believed to comprise cancerous tissue to assess one of the spatial location, the grade or the character of the cancerous tissue, comprising:
illuminating the area of interest with electromagnetic radiation (emr) having at least one wavelength of from 450 nm to 2500 nm;
administering a contrast enhancing agent to the patient;
detecting one or more optical properties of a plurality of spatially resolved areas in the area of interest following administration of the contrast enhancing agent and acquiring a data set representing the one or more optical properties corresponding to each of the spatially resolved areas of the area of interest;
comparing the optical properties of the spatially resolved areas within the area of interest in the acquired data set subsequent to administering the contrast enhancing agent to one of different spatially resolved areas of the area of interest and a control data set representing a corresponding one or more optical properties of tissue having an identified tissue type and condition; and
identifying and spatially locating pathological tissue in the area of interest based on differences between the acquired data set and the control data set.

22. The method of claim 21, for screening a tissue sample selected from the group consisting of: breast tissue; uterine tissue; cervical tissue; intestinal tissue; colorectal tissue; esophageal tissue; skin; prostate tissue; lymph tissue; bone; and brain tissue.

23. The method of claim 21, additionally comprising positioning an optical source and an optical detector for epi-illumination of the area of interest.

24. The method of claim 21, additionally comprising positioning an optical source and an optical detector for transillumination of the area of interest.

25. An optical system for in situ detection of a physiological property of an area of interest comprising:
at least one optical source for illuminating an area of interest believed to contain tissue having the physiological property with electromagnetic radiation (emr) having at least one wavelength of from 450 nm to 2500 nm, wherein the area of interest is located within a patient;
an optical source controller in communication with the at least one optical source for controlling the at least one optical source;
at least one optical detector for detecting and acquiring a data set representing one or more optical properties of tissue in spatially resolved areas within the area of interest;
an optical detector controller in communication with the at least one optical detector for controlling the at least one optical detector;
a central data processing unit in communication with the optical source controller and the optical detector controller for receiving the data set from the optical detector(s), comparing the acquired data set with a control data set not derived from the area of interest, and producing output data identifying differences in the acquired data set and the control data set; and
a display unit for displaying the output data provides control and adjustment of the timing of administration of the contrast enhancing agent.

26. The optical system of claim 25, wherein tissue is believed to contain cancerous tissue.

27. The optical system of claim 25, wherein the tissue is believed to contain at least one of abnormal cortical conditions, abnormal intracranial conditions, central or peripheral nervous system activity, or neuronal activity

28. An optical system for in situ detection of at least one of abnormal cortical conditions, abnormal intracranial conditions, central or peripheral nervous system activity, or neuronal activity, comprising:
at least one optical source for illuminating an area of interest comprising central or peripheral nervous system or neuronal tissue with electromagnetic radiation (emr) having at least one wavelength of from 450 nm to 2500 nm;
an optical source controller in communication with the at least one optical source for controlling the at least one optical source;
at least one optical detector for detecting and acquiring a data set representing one or more optical properties of central or peripheral nervous system or neuronal tissue in spatially resolved areas within the area of interest;
an optical detector controller in communication with the at least one optical detector for controlling the at least one optical detector;
a central data processing unit in communication with the emr source controller and the optical detector controller for receiving the data set from the optical detector(s), comparing differences in the optical properties of the spatially resolved areas within the area of interest, and producing output data identifying differences in optical properties in the spatially resolved areas within the area of interest; and
a display unit for displaying the output data.

29. A method for spatially mapping blood flow and blood vessels in an area of interest, comprising:
illuminating an area of interest including a blood vessel with electromagnetic radiation (emr) having at least one wavelength of from 450 nm to 2500 nm;
administering an external effector comprising at least one of a stimulus and a contrast enhancing agent to the area of interest;
detecting one or more optical properties of the area of interest following administration of the stimulus or the contrast enhancing agent and acquiring a data set representing the one or more optical properties of spatially resolved areas within the area of interest; and
comparing differences in the optical properties of spatially resolved areas within the area of interest, whereby differences in the optical properties are characteristic of differences of blood flow at spatially resolved areas within the area of interest,
wherein the illuminating or detecting is performed external to the blood vessel.

30. The method of claim 29, wherein the illumination or detection steps are performed non-invasively.

31. The method of claim 29, wherein the illuminating or detecting is performed external to the blood vessel.

32. The method of claim 29, wherein the area of interest is located in a patient and the method additionally comprises positioning one or more optical source and detector pairs in non-invasive contact with the patient prior to administering the external effector.

33. The method of claim 29, wherein the area of interest is located in a patient and the method additionally comprises positioning one or more optical source and detector arrays in non-invasive contact with the patient prior to administering the external effector.

34. The method of claim 29, additionally comprising positioning at least one optical source and at least one optical detector for epi-illumination of the area of interest.

35. The method of claim 29, additionally comprising positioning at least one optical source and at least one optical detector for transillumination of the area of interest.

36. The method of claim 29, wherein the illuminating is from an optical source and detecting is from a separate detector.

37. A method for detecting an area of abnormal blood flow and abnormal blood vessels in an area of interest, comprising:
illuminating an area of interest including a blood vessel with electromagnetic radiation (emr) having at least one wavelength of from 450 nm to 2500 nm;
detecting one or more optical properties in spatially resolved areas within the area of interest and acquiring a data set representing the one or more optical properties of spatially resolved areas within the area of interest including the blood vessel; and
comparing the data set to a control data set representing a corresponding optical property of spatially resolved areas having a normal blood flow and identifying differences in the data set and the control data set, whereby differences in the optical properties of the data set at spatially resolved areas within the area of interest compared to the control data set are indicative of abnormalities in blood flow.

38. The method of claim 37, wherein the illumination or detection steps are performed non-invasively.

39. The method of claim 37, wherein the illuminating or detecting is performed external to the blood vessel.

40. The method of claim 37, wherein the illuminating is from an optical source and detecting is from a separate detector.

41. The method of claim 37, wherein the control data set represents one or more corresponding optical properties of spatially resolved areas empirically determined to be indicative of normal blood flow.

42. The method of claim 37, wherein the illuminating is from an optical source and detecting is from a separate detector.

43. An optical system for in situ detection of at least one of abnormal blood flow, blood accumulation and abnormal blood characteristics in an area of interest, comprising:
an optical source for illuminating the area of interest including at lest one blood vessel with emr having at least one wavelength of from 450 nm to 2500 nm;
an optical detector for detecting and acquiring a data set representing optical properties of spatially resolved areas within the area of interest;
a data processing unit for receiving the data set from the optical detector, comparing differences in the optical properties of the spatially resolved areas within the area of interest, and producing output data identifying differences in the optical properties of the spatially resolves areas within the area of interest, whereby differences in the optical properties are representative of at least one of blood vessels, blood flow and blood characteristics in the area of interest; and
a display unit for displaying the output data.

44. The optical system according to claim 43, further for spatial mapping the blood vessels, blood flow or blood characteristics and further including spatial mapping and an optical detector controller in communication with the at least one optical detector for controlling the at least one optical detector.

45. The optical system according to claim 43, wherein the at least one optical source or at least one detector is located external to the blood vessel.

46. An optical system according to claim 43, wherein the optical sources comprises an array of optical sources and the optical detectors comprises an array of optical detectors separately located from the optical sources.

47. An optical system according to claim 43, wherein the at least one optical source and the at least one optical detector are mounted non-invasively.
